(19) United States
(12) Patent Application Publication
(10) Pub. No.: US 2021/0252226 A1
(43) Pub. Date: Aug. 19, 2021

(54) Pharmaceutical Product Preparation Device and Method

(71) Applicant: Janssen Pharmaceuticals, inc., Titusville, NJ (US)

(72) Inventors: Paul Tashjian, Phoenixville, PA (US); Emma Richard, Conshohocken, PA (US)

(21) Appl. No.: 16/973,182

(22) PCT Filed: Jun. 14, 2019
(86) PCT No.: PCT/IB2019/055000

§ 371 Date: Dec. 8, 2020

(30) Foreign Application Priority Data
Jun. 14, 2018 (GB) 1809776.6

(51) Int. Cl. A61M 5/315; A61M 5/31; A61M 5/32

(52) U.S. Cl. CPC A61M 5/31501; A61M 5/31513; A61M 2005/3114; A61M 5/3202; A61M 5/3137

(57) ABSTRACT

A syringe is disclosed. The syringe comprises a body and a plunger which is moveable between an initial position and a retracted position within, and relative to, the body, wherein the body and plunger define a variable cavity volume within the syringe. The syringe also comprises a locking member configured to lock the plunger in its retracted position relative to the body. Methods and systems for reconstituting a pharmaceutical product are also disclosed. A flexible seal that is fixed to a proximal region of the body of the syringe and has an expanded stretched configuration and a relaxed contracted configuration is also disclosed. The flexible seal may be fixable to a proximal region of the body of the syringe via an end cap, which is configured for removable or fixed attachment to the proximal end of the body.

### TECHNICAL FIELD

The present invention relates generally to the field of administration of a pharmaceutical product, and more specifically to the field of devices and methods for the reconstitution and/or administration of a pharmaceutical product.

### BACKGROUND

The administration of pharmaceutical products to human or animal patients occurs daily in modern health and veterinary care. A common form of administration is administration via a syringe, whereby a pharmaceutical product is injected into a patient.

Prior to administration, the pharmaceutical product must be prepared. Whilst some pharmaceutical products are able to be stored long term in a state suitable for administration, certain pharmaceutical products require preparation immediately before use, which involves mixing a first component of the pharmaceutical product to be prepared with the second component of the pharmaceutical product to be prepared, in order to form a reconstituted or mixed pharmaceutical product. The first and second components may be fluid or solid, but once mixed form a fluid that may be administered to a patient, for example by a syringe.

Current methods for preparing a syringe of a mixed pharmaceutical product from its constituent parts involve a large number of steps where connections are made between vials, needles and syringes. Typical methods include opening and sterilising two vials, containing a first component of the mixed pharmaceutical product (such as a drug) and a second component of the mixed pharmaceutical product (such as water for injection) respectively. A preparation (“prep”) syringe is then prepared with a needle attached. The needle is uncapped and inserted into the vial containing the water for injection (WFI), which can then be drawn into the prep syringe. The prep syringe is then withdrawn from the WFI vial, and inserted into the vial containing the drug. The WFI is then injected by the prep syringe into the drug vial. The prep syringe is then withdrawn from the vial, and the drug is left to dissolve in the WFI.

When the mixed pharmaceutical product has been prepared, a needle of a transfer syringe is uncapped and inserted into the drug vial, from where the mixed pharmaceutical product can be drawn into the transfer syringe. Once the mixed pharmaceutical product has been drawn into the transfer syringe, the needle is recapped, and the syringe of prepared mixed pharmaceutical product is ready for use.

The large number of steps involved produces numerous opportunities for needle sticks, contamination and connection issues. In addition, the repetition of steps may be prone to user error and causes user fatigue, particularly in preparation of large amounts of reconstituted drug. Current methods also require the use of numerous pieces of apparatus, making storage of the components and apparatus required to prepare a mixed pharmaceutical product difficult and inefficient.

A more recent addition to the art is dual chamber devices. In these devices, a syringe is provided which contains, in separated chambers, multiple components of a mixed pharmaceutical product to be mixed. These devices include mechanisms by which the multiple components can be mixed before use. However, these devices are expensive to produce. These devices also face issues of insufficient vapour barriers between the chambers, and there is a risk of barrier breakdown during transit or storage, which would render the device and the components of the mixed pharmaceutical product unusable.

### SUMMARY

A first aspect of the invention relates to a syringe. The syringe comprises a body, a plunger moveable between an initial position and a retracted position within and relative to the body, and a locking mechanism configured to lock the plunger in the retracted position relative to the body, wherein the body and plunger define a cavity volume within the syringe. The volume or capacity of the cavity volume is variable, according to the relative position of the position of the plunger within the body. By this mechanism, a decreased pressure can be created and maintained within a defined cavity volume of a specific volume, allowing a desired volume of fluid to be drawn into the cavity volume. The specific volume may be determined according to factors such as required dosage, or a desired ratio of a first component of a mixed pharmaceutical product (which may be provided in the cavity volume when the plunger is in the initial position) to a second component of a mixed pharmaceutical product (which may be the fluid to be drawn into the cavity volume by the created decreased pressure).

The body of the syringe may comprise a discharge opening at a distal end and a plunger receiving opening at a proximal end.

The retracted position may be one in which the plunger is retracted proximally relative to the initial position. When the plunger is in this retracted position, the defined cavity volume will be increased relative to the cavity volume defined by the body and the plunger when the plunger is in the initial position.

The plunger may be further moveable to a depressed position, wherein the depressed position is one in which the plunger is depressed distally relative to the initial position. When the plunger is in this depressed position, the defined cavity volume will decreased relative to the cavity volume defined by the body and the plunger when the plunger is in the initial position.

The syringe may further comprise an automatic retraction stop mechanism configured to prevent the plunger from being moveable to a position proximal of the retracted position. This automatic retraction stop mechanism prevents the plunger from being pulled out of the body of the syringe. The automatic retraction stop may also prevent the plunger from being overly retracted to define a cavity volume which exceeds the specific volume, and thereby may prevent fluid in excess of the desired volume being drawn into the cavity volume.

A portion of the plunger may create an air tight seal with the interior of the syringe body, such that the decreased pressure within the cavity volume is created within the cavity volume when the plunger is moved proximally. In some embodiments, the portion of the plunger that creates an air tight seal with the interior of the syringe body comprises rubber.

In some embodiments, the locking mechanism may comprise a latching member, extendable from the plunger to latch against a portion of the body of the syringe when the plunger is in the retracted position, thereby locking the plunger in the retracted position relative to the body. The locking mechanism may further comprise a biasing member adapted to extend the latching member, the latching member being configured to extend laterally outwards of the plunger and to latch against the portion of the body of the syringe when the plunger is withdrawn to the retracted position and upon exertion of force from the biasing member on the latching member. This allows for automatic locking of the plunger in the retracted position, such that a user is not required to determine when the plunger has been retracted to a position which defines the required cavity volume, or to actively engage a lock. This configuration also enables the defined cavity volume to be maintained at the specific volume despite forces caused by the decreased pressure within the cavity volume acting on the plunger.

The portion of the body of the syringe, against which the latching member is configured to latch when the plunger is in the retracted position, may be formed on a flange located at a proximal end of the body. The flange of the body may form a finger support surface for supporting fingers of a user. In some embodiments, the body may comprise an opening through which the latching member is configured to extend when the plunger is withdrawn to the retracted position. Depression of the latching member laterally toward the plunger may unlock the plunger from the retracted position.

In alternative embodiments, the locking mechanism may comprise a latching member extendable from the body of the syringe to latch against a portion of the plunger when the plunger is in the retracted position, thereby locking the plunger in the retracted position relative to the body. The locking mechanism may further comprise a biasing member adapted to extend the latching member, the latching member being configured to extend laterally inwards of the plunger and to latch against the portion of the plunger when the plunger is withdrawn to the retracted position and upon exertion of force from the biasing member on the latching member. The plunger may further comprise an opening through which the latching member is configured to extend when the plunger is withdrawn to the retracted position. Depression of the latching member laterally outwards of the plunger may unlock the plunger from the retracted position.

The body of the syringe may further comprise a proximal end having at least one flange, and the latching member may be extendable from within the at least one flange. The flange of the body may form a finger support surface for supporting fingers of a user.

In some embodiments, the latching member may comprise a bar having a proximal end and a distal end, wherein the proximal end is pivotably secured within a proximal portion of the plunger and the distal end is configured to extend laterally outwards of the plunger and to latch against a portion of the body of the syringe. This provides for a large surface area on which a user or other component of the syringe may act to depress the latching member to unlock the plunger from the retracted position.

In further embodiments, the plunger may include a button which is movable between an actuated position and a non-actuated position, the button being configured to disengage the latching member from the portion of the body of the syringe against which the latching member latches when the button in the actuated position. The button may be located on any portion of the plunger which is accessible to a user when the plunger is in the retracted position. The button may be provided at a more easily accessible position on the plunger than the latching member, and may therefore provide an ergonomic way for a user to unlock the plunger from the retracted position. The button may also allow for increased or decreased force to be required to unlock the plunger from the retracted position than is actually required to overcome the force provided by the biasing member onto the latching member. This could be helpful should the syringe be intended for use by someone with reduced hand strength or agility, or should it be beneficial for accidental unlocking of the plunger from the retracted position to be hindered.

In further embodiments, the latching member may comprise a camming surface and the button may be configured, when actuated, to act against the camming surface to move the latching member in a direction laterally inwards of the plunger.

In alternative embodiments, the latching member comprises teeth and the plunger further comprises a gear wheel configured to engage the teeth, wherein actuation of the button is configured to cause rotation of the gear wheel in a direction which drives the latching member to retract in a direction laterally inwards of the plunger.

In further embodiments, the button may be configured to reside in the actuated position when the plunger is in the initial position and the button is driven to the non-actuated position when the plunger is withdrawn to the retracted position. This driving of the button to the non-actuated position may be upon force from the biasing member. Transition of the button from the actuated position to the non-actuated position can provide an indication that the locking mechanism has successfully and fully locked the plunger in the retracted position.

In alternative embodiments, the plunger may further comprise an interior member which extends distally from the button and is positioned to be abutted by an end of the biasing member which is opposite to the latching member when the button is in the non-actuated position, the interior member being configured to move longitudinally through the plunger concurrently with the button, and the interior member comprising an opening which is larger than the abutting face of the biasing member and located at a position corresponding to that of the biasing member when the button is in the actuated position. The opening may extend partially or entirely through the interior member, provided it is deep enough to allow the biasing member to extend into it sufficiently for the latching member to disengage the syringe body.

In some embodiments, the plunger may comprise a cylindrical body portion comprising an opening through which the latching member is configured to extend laterally when the plunger is withdrawn to the retracted position and upon force from the biasing member. This cylindrical body may provide additional structural support to the plunger. The cylindrical body may also include an indication that the plunger has been retracted to the retracted position, thereby removing any uncertainty that the device may have jammed rather than the plunger having reached the fully retracted position.

One or both of the body and the plunger may be configured, when the plunger is in the initial position or the depressed position, to prevent the latching member from extending in a direction laterally outwards of the plunger.

The syringe may be provided an initial state with the plunger in the initial position, wherein the cavity volume of the syringe holds a first component of a mixed pharmaceutical product.

The syringe may further be provided in a second state with the plunger in the retracted position, wherein the cavity volume of the syringe holds a first component of a mixed pharmaceutical product and a second component of the mixed pharmaceutical product which has been drawn into the cavity volume via the discharge opening in response to retraction of the plunger from the initial position to the retracted position.

The syringe may be configured to expel the mixed pharmaceutical product from the cavity volume via the discharge opening in response to depression of the plunger from the retracted position towards the depressed position.

In embodiments in which a portion of the plunger creates an air tight seal with the interior of the syringe body, this portion of the plunger may comprise a flexible seal coupled to a proximal region of the body of the syringe. This flexible seal can have an expanded configuration and a contracted configuration. The flexible seal may be configured to relax, contract, compress, collapse or otherwise transition from the expanded configuration to the contracted configuration as the plunger is moved proximally, from the initial position towards the retracted position, and/or the flexible seal may be configured to stretch or expand from the contracted configuration to the expanded configuration as the plunger is moved distally, from the retracted position towards or past the initial position. The flexible seal provides for the maintenance of a sterile pathway within the cavity volume.

An entire circumference of the flexible seal may be coupled to or within the body of the syringe. In some embodiments, a central portion of the flexible seal may be coupled to a distal end of a piston of the plunger. This fixation can help to ensure approximately symmetrical transitioning, stretching, expansion, compression, contraction, etc. of the flexible seal around a piston of the plunger as the plunger moves between the retracted, initial, and depressed positions.

In some embodiments, an internal surface of the body of the syringe, and a distal end of the plunger each have a correspondingly rounded shape. In some embodiments, the internal surface of the body of the syringe, and the distal end of the plunger each have a correspondingly hemi-ellipsoidal shape, which may be hemispherical. These configurations of syringe body interior and plunger distal end provide an ideal chamber seal, and provide a smooth surface over which the flexible seal may stretch, expand, compress, contract, or otherwise transition. This can allow for thinner or less sturdy materials to be used for the flexible seal, since points at which pressure to a stretched or stretching flexible seal (by, for example, a sharpened edge on a piston of the plunger) may otherwise be concentrated, are minimised or removed.

In some embodiments, the flexible seal is coupled to a proximal end cap which is attached to the body at a proximal end of the body. The end cap may be configured for removable or fixed attachment to the body.

In some embodiments, the flexible seal conforms to the shape of the distal end of the piston when the plunger is positioned in or between the retracted position and the depressed position, and/or an expanded portion of the flexible seal conforms to the shape of the internal surface of the distal end of the body when the plunger is in the depressed position.

Another aspect of the present invention is a method of reconstituting a mixed pharmaceutical product. The method includes the steps of a) providing a syringe having a body, a plunger moveable between an initial position and a retracted position relative to the body, a discharge opening, a locking mechanism configured to lock the plunger in the retracted position, and a cavity volume defined by the body and plunger, wherein the plunger is provided in the initial position, and the cavity volume defined by the body and the plunger in the initial position contains a first component of the mixed pharmaceutical product, b) placing the discharge opening into fluid communication with a vial containing a second component of the mixed pharmaceutical product, c) retracting the plunger to draw in, or commence drawing in, of the second component of the mixed pharmaceutical product into the cavity volume, and d) upon full retraction of the plunger, the locking mechanism engaging. In this method, the second component continues to be drawn in while the locking mechanism is engaged, until the cavity volume defined by the body and the plunger in the retracted position is filled or the drawing in of the second component of the mixed pharmaceutical product into the cavity volume defined by the body and the plunger in the retracted position is terminated. This method allows for the reconstitution of a mixed pharmaceutical product using fewer pieces of apparatus and in less steps than by conventional methods, and avoids the risk of premature mixing of portions of the components, which is present in dual chamber devices. By requiring fewer steps and pieces of apparatus, the method provides further benefits in reducing the risk of contaminants, eliminating agglutination and additional fluid dynamic shear on molecules, reducing occasions where there is a risk of needle stick injury to a user, and minimising user fatigue by requiring less repetitive steps and providing a faster and more efficient method.

In some embodiments, the method may further include unlocking the locking mechanism after the second component of the mixed pharmaceutical product has been drawn into the cavity volume defined by the body and the plunger in the retracted state. The locking mechanism may comprise a latching member and a biasing member, and unlocking the locking mechanism may comprise a user pushing the latching member toward the longitudinal axis of the syringe. In further embodiments, the plunger may comprise a button configured to engage the locking mechanism, and unlocking the locking mechanism may comprise a user actuating the button.

The method may further include shaking the syringe after the second component of the mixed pharmaceutical product has been drawn into the cavity volume. The shaking may occur before, after or without unlocking the locking mechanism. Shaking improves reconstitution of the drug. The locking mechanism being engaged while syringe is shaken helps to prevent loss of the mixed pharmaceutical product, or components thereof, by accidental depression of the plunger.

In some embodiments, the method may further include removing the discharge opening from fluid communication with the vial and replacing a component of the discharge opening after the second component of the mixed pharmaceutical product has been drawn into the cavity volume defined by the body and the plunger in the retracted position. The component of the discharge opening which is replaced may be a needle or transfer member. Replacing this element after the second component of the mixed pharmaceutical product has been drawn into the cavity reduces the risk of contaminants from the vial, or otherwise, being transferred. It also reduces the risk of blockage by stopper coring, and the risk of traces of the second component (which may reside in the first needle or transfer member when the mixed pharmaceutical product within the cavity volume is reconstituted) from being transferred.

Another aspect of the invention is a system for reconstituting a mixed pharmaceutical product. The system comprises a syringe comprising a body, a plunger moveable between an initial position and a retracted position relative to the body, a discharge opening, a locking mechanism configured to lock the plunger in the retracted position, and a cavity volume defined by the body and plunger, wherein the cavity volume contains a first component of the mixed pharmaceutical product. The system further comprises a vial containing a second component of the mixed pharmaceutical product. In a further embodiment, the system comprises any syringe described herein.

The first component of the mixed pharmaceutical product may be in solid form. The first component of the mixed pharmaceutical product may be in the form of a lyophilized powder, a lyophilized cake, or lyophilized granules. Provision of the syringe in the initial state, holding such a first component allows for convenient storage of a component of a mixed pharmaceutical product to be reconstituted, and a reduction in the amount of apparatus required. It also reduced the number of occasions on which a user is exposed to the risk of needle stick injuries. The second component of the mixed pharmaceutical product may be a liquid, such as sterilised water. The mixed pharmaceutical product may be reconstituted Remicade (RTM). The mixed pharmaceutical product may be Sylvant (RTM).

Another aspect of the invention is a syringe which comprises a body, and a plunger moveable between an initial position and a retracted position within and relative to the body, wherein the body and the plunger define a cavity volume within the syringe. The plunger may comprise a piston, and a flexible seal coupled to a proximal region of the body of the syringe and having an expanded configuration and a contracted configuration. The flexible seal may be configured to transition from the expanded configuration to the contracted configuration as the plunger is moved proximally, from the initial position towards the retracted position, and/or the flexible seal may be configured to expand from the contracted configuration to the expanded configuration as the plunger is moved distally, from the retracted position towards or past the initial position. In some embodiments, the flexible seal is configured to stretch from the contracted configuration to the expanded configuration, and relax from the expanded configuration to the contracted configuration. The flexible seal provides for the maintenance of a sterile pathway within the cavity volume.

An entire circumference of the flexible seal may be coupled to or within the body of the syringe. In some embodiments, a central portion of the flexible seal may be coupled to a distal end of the piston of the plunger. This fixation can help to ensure approximately symmetrical transitioning, stretching, expansion, compression, contraction, etc. of the flexible seal around a piston of the plunger as the plunger moves between the retracted, initial, and depressed positions.

In an embodiment, the flexible seal is attached to a proximal end cap which is attached to the body at a proximal end of the body. The proximal end cap may be configured for removable or fixed attachment to the body.

In some embodiments, an internal surface of the body of the syringe, and a distal end of the plunger each have a correspondingly rounded shape. In some embodiments, the internal surface of the body of the syringe, and the distal end of the plunger each have a correspondingly hemi-ellipsoidal shape, which may be hemispherical. These configurations of syringe body interior and plunger distal end provide an ideal chamber seal, and provide a smooth surface over which the flexible seal may stretch, expand, compress, contract, or otherwise transition. This can allow for thinner or less sturdy materials to be used for the flexible seal, as points at which pressure to a stretched or stretching flexible seal (by, for example, a sharpened edge on a piston of the plunger) may otherwise be concentrated, are minimised or removed.

In some embodiments, an expanded portion of the flexible seal conforms to the shape of the distal end of the piston when the plunger is positioned in or between the retracted position and the depressed position, and/or the flexible seal conforms to the shape of the internal surface of distal end of the body when the plunger is in the depressed position.

In some embodiments, the syringe may further comprise a locking mechanism, such as any of the locking mechanisms described herein, configured to automatically engage when the plunger is withdrawn to the retracted position to lock the plunger in the retracted position relative to the body, to prevent at least distal movement of the plunger from the retracted position.

The locking mechanism may comprise a latching member extendable from the plunger to latch against a portion of the body of the syringe when the plunger is moved to the retracted position, thereby locking the plunger in the retracted position relative to the body. The portion of the body of the syringe against which the latching member latches may be proximal of the portion of the body to which the flexible seal is fixed.

The locking mechanism may comprise a latching member extendable from the body of the syringe to latch against a portion of the plunger when the plunger is in the retracted position, thereby locking the plunger in the retracted position relative to the body. The latching member may extend from the body proximal of the portion of the body to which the flexible seal is fixed.

Another aspect of the invention is an end cap for attachment to a proximal end of a body of a syringe. The end cap comprises a connector adapted for attaching the end cap to the proximal end of the body, an aperture formed therethrough, sized to movably receive a piston of a plunger, and a flexible seal attachable to the end cap and covering the aperture. The flexible seal has an expanded configuration and a contracted configuration, and the flexible seal is configured to transition from the expanded configuration to the contracted configuration within the body of the syringe, when the end cap is attached to the proximal end of the syringe. In some embodiments, the flexible seal is configured to stretch from the contracted configuration to the expanded configuration, and relax from the expanded configuration to the contracted configuration.

The end cap can be configured for removable or fixed attachment to the body. In some embodiments, the flexible seal is attached to or within the end cap. In some embodiments, the flexible seal is configured to be fixed between the end cap and the body, when the end cap is attached to the proximal end of the body.

### BRIEF DESCRIPTION OF THE FIGURES

The invention is described below with reference to the associated figures. The figures are illustrative and are not intended to define the exact scale of any of the features of the invention. Solid arrows denote directions, dashed lines generally denote parts which are obscured by other parts in the illustrated view, unless specified otherwise.

FIG. 1A is a side view of a syringe with the plunger in the initial position, and in which a first component of a pharmaceutical product to be mixed is contained.

FIG. 1B is a side view of the syringe of FIG. 1A, which has been placed in fluid communication with a vial containing a second component of the pharmaceutical product to be mixed. The plunger has been retracted to the retracted position and the locking mechanism has engaged.

FIG. 1C is a side view of the syringe of FIG. 1B, in which the plunger is in the retracted position and the locking mechanism has been disengaged.

FIG. 1D is a side view of the syringe of FIG. 1C, in which the plunger has been depressed to the depressed position.

FIG. 2 is a cutaway view of an embodiment of the syringe of FIG. 1, in which the locking mechanism is engaged.

FIG. 3 is a cutaway view of an alternative embodiment of the syringe of FIG. 1, in which the locking mechanism is engaged.

FIG. 4 is a cutaway view of an alternative embodiment of the syringe of FIG. 1, in which the locking mechanism is engaged.

FIG. 5 is a cutaway view of an alternative embodiment of the syringe of FIG. 4, in which multiple retracted positions of the plunger are defined, and in which the locking mechanism is engaged.

FIG. 6 is a cutaway view of an alternative embodiment of the syringe of FIG. 1, in which the locking mechanism is engaged.

FIG. 7 is a cutaway view of an alternative embodiment of the syringe of FIG. 2 which comprises a button. The locking mechanism is engaged and the button is in the non-actuated position.

FIG. 8 is a cutaway view of an alternative embodiment of the syringe of FIG. 4 which comprises a button. The locking mechanism is engaged and the button is in the non-actuated position.

FIG. 9 is a cutaway view of an alternative embodiment of the syringe of FIG. 1 which comprises a button. The locking mechanism is engaged and the button is in the non-actuated position.

FIG. 10 is a cutaway view of an alternative embodiment of the syringe of FIG. 2 which comprises a button. The locking mechanism is engaged and the button is in the non-actuated position.

FIG. 11A is a cutaway view of an alternative embodiment of the syringe of FIG. 1 which comprises a button. The locking mechanism is engaged and the button is in the non-actuated position.

FIG. 11B is a sectional view of the syringe of FIG. 11A, taken along the dashed line 11B of FIG. 11A.

FIG. 12 illustrates a method according to an embodiment of the present invention.

FIG. 13A is a side view of a syringe with an alternative plunger which comprises a flexible seal whereby the plunger is in the initial position and the flexible seal is expanded.

FIG. 13B is a side view of the syringe of FIG. 13A whereby the plunger has been positioned in the retracted position and the locking mechanism is engaged, and the flexible seal has contracted to a contracted configuration.

FIG. 14 is a side view of an alternative syringe and plunger, which also comprises a flexible seal, whereby the plunger is in the initial position and the flexible seal is expanded.

FIG. 15A is a side view of a syringe which comprises an end cap, attachable to the proximal end of the syringe body and which provides at least the flexible seal. The plunger is in the initial position and the flexible seal is expanded.

FIG. 15B is a side view of the syringe of FIG. 15A whereby the plunger has been positioned in the retracted position and the locking mechanism is engaged, and the flexible seal has been allowed to contract to a contracted configuration.

### DETAILED DISCLOSURE

The following detailed disclosure outlines the features of embodiments of the present invention. In addition, some (but by no means all) variants of the disclosed embodiments that might be implemented whilst still falling under the scope of the present invention are also described. Whilst the following description is subdivided into sections in order to aid the skilled person's comprehension, the specific substructure of the detailed description should not be seen as delimiting individual embodiments of the invention. On the contrary, features of the various sections may be combined as appropriate.

Reference to the features shown in the figures described below can be made in order to understand the principles of the present invention.

Common Features and Definitions

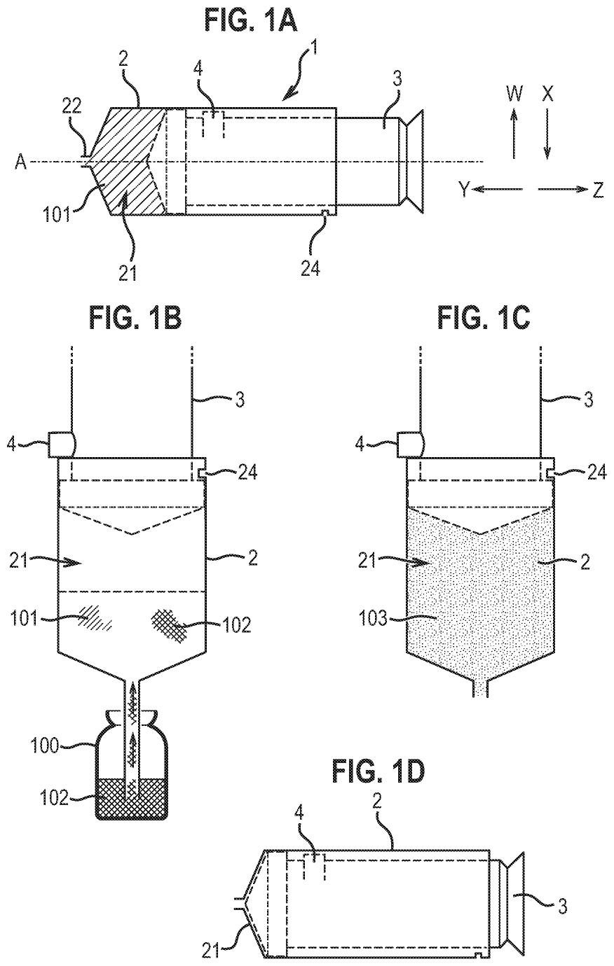

The longitudinal axis of the syringe is used herein to define the axis along which the plunger is moveable, and is shown in FIG. 1A by axis A. The lateral direction is considered to be any direction perpendicular to the longitudinal axis. The direction laterally outwards of the plunger is shown, in FIG. 1A, by arrow W, and the direction laterally inwards of, or towards, the plunger is shown, in FIG. 1A, by arrow X.

By “distal direction” is meant a direction along the line from, and preceding, the proximal end to, and extending beyond, the distal end of the syringe. The distal direction is shown, in FIG. 1A, by arrow Y. By “proximal direction” is meant a direction along the line from, and preceding, the distal end to, and extending beyond, the proximal end of the syringe. The proximal direction is shown, in FIG. 1A, by arrow Z.

An “opening” is a hole or space in a part, through which another part may pass or be dispensed. An opening may be of any size or shape, and may be considered to point in a direction which is normal to the plane of the opening. A “discharge opening” may, but is not limited to, include a needle or transfer member, or a mechanism by which a needle or transfer member may be releasably attached.

A “transfer member” is a structure able to operate as the structural connection between two fluidly coupled parts. The transfer member thereby provides a fluid pathway between the two parts.

By “air tight seal” is meant a seal which substantially prevents the flow of air across the sealing portion. The term is not intended to limit to seals which prevent air flow across the sealing portion entirely.

By describing an action as “automatic” is meant that the action occurs and may be completed without further manual intervention. The action may be initiated by manual intervention and then proceed automatically. Further, a first action may be initiated, proceed automatically and by virtue of the first action proceeding partway or to completion, an automatic initiation of a second action may also occur and by this mechanism, the second action is ultimately initiated by initiation of the first action.

By describing a feature as “automatic” is meant that the feature acts to perform a function for which it is configured without manual intervention.

A “locked state” as used herein generally refers to a state of a feature in which the plunger is prevented from moving.

An “unlocked state” as used herein generally refers to a state of a feature in which the plunger is able to move.

A “bar” may be fabricated of any suitable material, and is not limited to having a homogeneous cross-section.

A “camming surface” is intended to include any surface of a part against which another part can act to transfer motion, and is not limited to an external surface of the part.

The terms “initial position”, “retracted position”, and “depressed position” are utilised for ease of reference and are not necessarily intended to define a specific chronological order.

The term “mixed pharmaceutical product”, as used herein, refers to a product made of at least two components, a first component of a mixed pharmaceutical product 101 and a second component of a mixed pharmaceutical product 102. The process of mixing the pharmaceutical product may be to reconstitute the mixed pharmaceutical product prior to administration to the patient. The components may be of the mixed pharmaceutical product Remicade (RTM) and may be sterilised water and powdered Remicade (RTM) (INN: infliximab). Nevertheless, the components may be for a different mixed pharmaceutical product without affecting the method of the present invention or the operation of the device of the present invention. The components may be of the mixed pharmaceutical product Sylvant (RTM) (INN: siltuximab).

By “fluid communication” is meant a structural connection between two or more parts, permitting the transfer of fluid therebetween. The term only means that a fluid pathway has been established, and does not necessarily imply that a transfer of fluid is actually occurring.

A “vial” is a part able to act as a receptacle, temporarily or permanently, for another part. For example, a vial may be used as a receptacle for a part of the mixed pharmaceutical product. A vial may, in the present invention, be interchangeable with a jar, ampule, cylinder, packet, bottle or any other suitable receptacle.

The term “drawing in” is generally used herein to refer to the movement of a fluid under the influence of a decreased pressure, wherein a decreased pressure may refer to one or both of a pressure less than atmospheric pressure, or a pressure within a part which is less than the pressure within that part momentarily beforehand.

As used herein, “shaking” of a part refers to the periodic or non-periodic agitation/stirring of a part by manual or automatic means in order to encourage movement of the part. When the part is a component of a pharmaceutical product to be reconstituted, the shaking creates larger interaction surfaces for the component, thereby aiding rapid completion of the reconstitution process.

Vials may contain an external scale indicating the volume or capacity of the vial, which can be read by a user to indicate the progress of filling or emptying the vial.

The vials will typically be sterile, and contain an opening, closed with a closure. The closure may be one or more septa, but alternative closures that are not septa may be used.

The vials may also include temporary protective seals, such as a plastic cover or a foil, to ensure the surface of the closure remains sterile prior to use.

The septa or protective seals may be removable by a user prior to use, or may be pierceable by a discharge opening, needle, transfer member, or any other suitable part associated with the syringe.

It is understood that any vials may be sold separately from the syringe, but that a syringe may also be sold with the vials as a kit.

Syringes and vials according to the present invention have a range of sizes, determined principally by the yield of mixed pharmaceutical product that is required for administration. Ergonomic use of the syringe of the present invention may also be a factor which determines the size of syringes and vials according to the invention.

### Structure

According to an aspect of the present invention, syringe 1 comprises a body 2, and a plunger 3 which is moveable between an initial position, shown in FIG. 1A, and a retracted position, shown in FIG. 1B. Body 2 and plunger 3 define a cavity volume 21 within the syringe. The volume of the cavity volume 21 is variable, according to the relative position of the position of the plunger within the body. Syringe 1 has a generally cylindrical shape. Body 2 has a generally cylindrical shape. The proximal end of body 2 may comprise an opening to receive the plunger. The distal end of body 2 may include an approximately frustoconical shape, terminating at its distal end with a discharge opening 22. The proximal end of body 2 may further comprise one or more finger support surfaces for supporting one or more of a user's digits when the user is moving the plunger distally from its retracted position. The retracted position of plunger 3 may be defined by the position of the plunger at which a specific and desired volume of the cavity volume is defined by the body and plunger.

The discharge opening 22 may include, or be configured to secure, a needle or transfer member through which fluid can pass, to provide a leak-free fluid path. The discharge opening may comprise a Luer connection, e.g. an ISO Luer connection, for universal connection to or with other medical devices, such as a needle, vial, drug container, tube etc. In some embodiments, the discharge opening 22 includes a female Luer connector. One advantage of providing a female Luer connector on the syringe is that the connector is standardised to be connected to many types of needles and transfer members, which commonly employ male Luer connectors. In alternative embodiments, a male Luer connector may be provided on the syringe. Alternative connectors other than Luer connectors may be used to secure a needle or transfer member to provide a leak-free fluid path. Alternatively, the discharge opening may itself comprise a needle, such as a hypodermic needle, for injection of syringe contents into the body.

Plunger 3 is shaped and sized to fit within the interior of body 2. The cross section of plunger 3 need not be homogeneous along its length. At least a portion of plunger 3 is configured to create an air tight seal with the interior of the syringe body 2, such that a decreased pressure created within cavity volume 21 when plunger 3 is moved proximally. The portion of plunger 3 which is configured to create an air tight seal with the interior of body 2 may comprise a sealing member. The sealing member may comprise any suitable feature, such as one or more O-rings. The portion of plunger 3 which is configured to create an air tight seal with the interior of body 2 may comprise a medical grade rubber, or elastomer which exhibits rubber like physical properties. At its proximal end, plunger 3 may include a surface to be pressed by a user to depress the plunger when unlocked.

The syringe may further comprise an automatic retraction stop mechanism 24, configured to prevent plunger 3 from being pulled out of the body of the syringe. The automatic retraction stop mechanism may prevent the plunger from being moveable to a position proximal of its retracted position. Alternatively, the automatic retraction stop mechanism may allow plunger 3 to be moveable to a position proximal of its retracted position, but prevent the plunger from being moveable proximal of a maximum capacity position, this maximum capacity position of the plunger being a position which is proximal of the retracted position of the plunger, relative to the initial position. As an example, the automatic retraction stop mechanism may comprise a ridge formed on the interior of the body of the syringe, the ridge being configured to engage a portion of the plunger, such as a proximal end of the sealing member, to prevent further proximal movement of the plunger. In alternative embodiments, the automatic retraction stop mechanism may be provided on one or both of body 2 and plunger 3. As will be discussed in later sections, the automatic retraction stop mechanism may be provided by the locking mechanisms of some embodiments of the present invention.

One or both of body 2 and plunger 3 may include markings thereon to denote the volume or capacity of the cavity volume defined by the body and plunger at a given position of the plunger. The markings may provide a user with an indication and/or confirmation that the plunger has reached, or is approaching, its retracted position. The markings may also be used to verify that the correct syringe is being used for the required dosage.

At least the body of the syringes described herein may comprise Borosilicate glass, to promote drug stability and lyophilization process compatibility. Additionally or alternatively, the body of the syringe may comprise a polymer material.

### Locking Mechanism

According to embodiments of the invention, syringe 1 includes a locking mechanism 4 configured to lock plunger 3 in its retracted position. When the plunger is retracted distally, the volume of cavity volume 21 is increased, and a decreased pressure is created within the cavity volume. This decreased pressure acts to draw fluid into the cavity volume when a fluid path is established with the cavity volume. The rate at which fluid enters the cavity volume depends upon various factors including, but not limited to, the rate of retraction of plunger 3, the viscosity of the fluid, and the size of the aperture through which the fluid path is established. As such, the rate at which fluid enters cavity volume 21 may be less than the rate at which the volume of the cavity volume is increased. The decreased pressure within the cavity volume also acts against the retraction of plunger 3. In syringes which are not provided with a locking mechanism 4, there is a risk that once the plunger has been retracted to its retracted position, the plunger will be forced distally as the cavity volume fills, until pressure equilibrium is reached between the cavity volume and the surrounding environment.

The locking mechanism of the present invention provides a solution to this forced distal movement of the plunger, and is configured to engage automatically when plunger 3 reaches its retracted position. This automatic engagement means that a user is not required to carefully observe for when the desired plunger position is reached, to hold the plunger position against the force of decreased pressure within the syringe, and/or or to manually engage a lock. This is beneficial in that the device is more suitable for use by those with reduced hand strength or dexterity, and may be used quickly effectively by both skilled health practitioners and others, if this is desired.

Locking mechanism 4 also enables efficient and effective reconstitution of a mixed pharmaceutical product according to a method of the present invention. Reconstitution of a mixed pharmaceutical product often requires the suspension of the first and second components of a pharmaceutical product to be mixed by shaking of the syringe, or to be left for some time to dissolve. During this step, a freely moveable plunger may be depressed accidentally, resulting in the discharging of fluid from the cavity volume. Such a discharge could result in an improper ratio of components in the product, or in a prepared dose which is less than that required, both of which could be harmful to a patient. Not only does the locking mechanism of the present invention make it easier to initially draw a specific, desired volume of a second component of a mixed pharmaceutical into a cavity volume of a syringe, the engagement of the locking mechanism when the plunger is in its retracted position ensures that no fluid is accidentally discharged from the cavity volume while the pharmaceutical product is mixed.

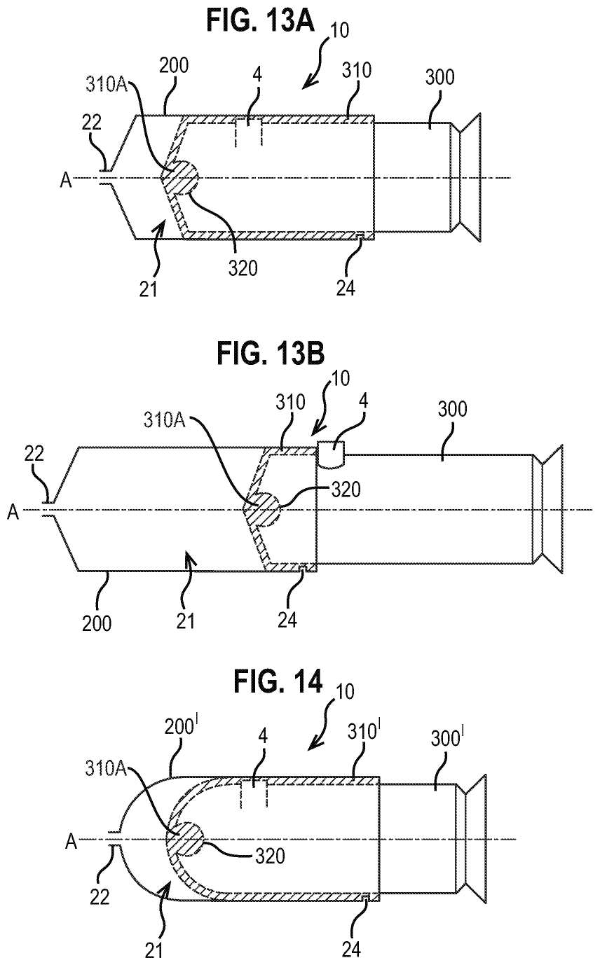

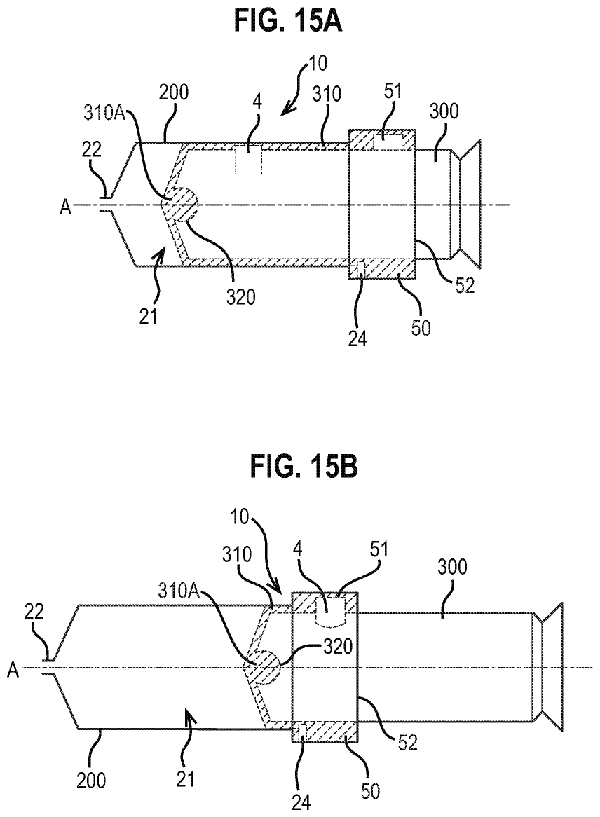

Whilst locking mechanism 4 is illustrated in FIGS. 1A to 1D in combination with syringe 1, it is also envisaged that locking mechanism 4 may be incorporated into syringes 10 and 10′, as illustrated in FIGS. 13A, 13B, and 14. Similarly, the variations on locking mechanism 4 which are described below may also be incorporated into syringes 10 and 10′.

### Push Switch Locking Mechanism

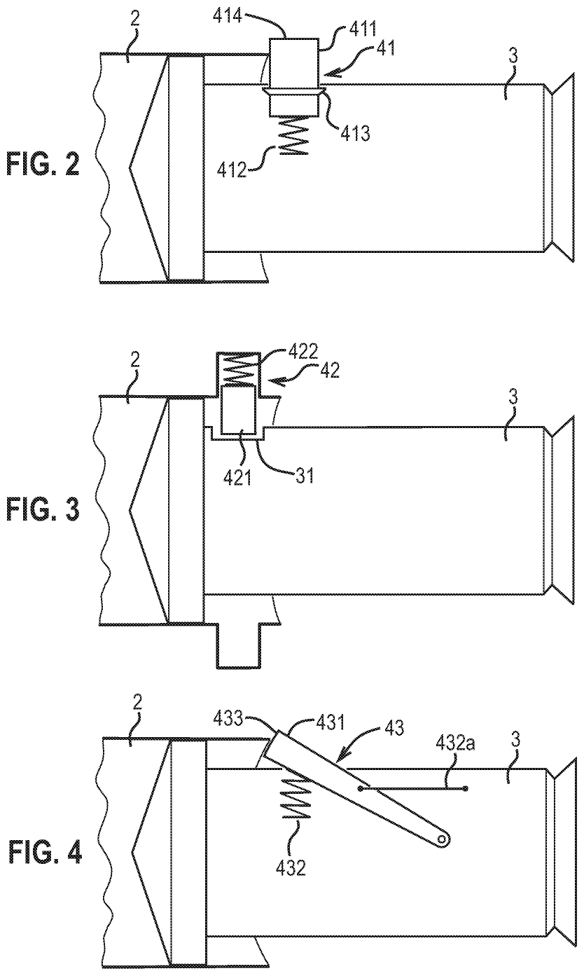

An exemplary locking mechanism 41 is illustrated in FIG. 2. Locking mechanism 41 comprises a latching member 411 and a biasing member 412. Biasing member 412 is configured to bias latching member 411 to extend laterally outwards of plunger 3 such that a portion of latching member 411 latches against a portion of body 2 when the plunger is in its retracted position, thereby preventing distal movement of plunger 3. Latching member 411 is positioned along the length of plunger 3 such that, when plunger 3 is positioned distally of its retracted position, latching member 411 is prevented from extending in a direction laterally outwards of plunger 3, for example, due to contact between the inner surface of the body 2 and the top surface of top switch 414.

The portion of body 2 which latching member 411 latches against may be a portion of the most distal end of the body. In an alternative embodiment, body 2 may include at least one flange formed at its proximal end, against which the latching member acts. The at least one flange may also provide a finger support surface for supporting fingers of a user operating the syringe when moving the plunger distally from its retracted position. In some embodiments, body 2 may include an opening at a location corresponding to the location of latching member 411 when plunger 3 is in its retracted position. The opening formed in body 2 is sized and shaped to receive latching member 411, and may surround the latching member when the plunger is in its retracted position and the latching member extends through the opening. In this configuration, the locking mechanism may prevent both distal and proximal movement of the plunger from its retracted position, and may therefore act as the automatic retraction stop mechanism. In an alternative embodiment, the opening may comprise a notch formed in the distal end of body 2.

In some embodiments, multiple retracted positions of plunger 3 may be defined, which correspond to multiple specific and desired volumes of cavity volume 21. In these embodiments, body 2 may include more than one opening, positioned at locations corresponding to the locations of latching member 411 when the plunger is in each of its retracted positions respectively.

Latching member 411 may include additional features, such as tabs 413, which limit the extent to which latching member 411 can extend laterally outwards of plunger 3. Alternatively, the extent to which the latching member can extend laterally outwards of the plunger may be limited by biasing member 412. Biasing member 412 is illustrated to be a compression spring, although alternative biasing members are envisioned, such as an elastomer or other resilient structure known in the art.

Locking mechanism 41 may be disengaged to allow for distal movement of plunger 3. A portion of latching member 411 may be configured as a push switch 414. In the illustrated embodiment, this portion is the surface of the latching member which faces outwards of the plunger, but may be any other suitable portion. To disengage the locking mechanism, a user may depress push switch 414, causing latching member 411 to retract in a direction laterally towards plunger 3, to a position in which the latching member does not latch against body 2 to prevent distal movement of the plunger.

In an alternative embodiment, shown in FIG. 3, the biasing member 422 and latching member 421 of locking mechanism 42 may be located substantially in body 2. In this embodiment, biasing member 422 is configured to bias latching member 421 to extend laterally inwards of the interior of body 2, such that a portion of the latching member latches against a portion of plunger 3, thereby preventing proximal movement of the plunger. Body 2 may also include at least one flange formed at its proximal end, from within which the latching is configured to extend when the plunger is in its retracted position. The at least one flange may also provide a finger support surface for supporting fingers of a user operating the syringe when moving the plunger distally from its retracted position. A portion of latching member 421 may be accessible by a user, for example a tab which extends outside of the flange, to disengage the locking mechanism. To disengage the locking mechanism, a user pulls or otherwise moves the tab, causing latching member 412 to retract in a direction laterally outwards of the plunger 3, to a position in which the latching member does not latch against plunger 3 to prevent distal movement of the plunger.

The latching member 421 is positioned along the length of the body such that, when the plunger is positioned distally of its retracted position, the latching member is prevented from extending laterally inwards of the interior of the body and latching against the plunger. This may be achieved by plunger 3 being substantially cylindrical along its length and including an opening or recessed portion 31 at a location corresponding to the location of the latching member when the plunger is in its retracted position (or multiple openings corresponding to the locations of the latching member when the plunger is in its each of its retracted positions respectively).

### Lever Locking Mechanism

An alternative exemplary locking mechanism 43 is illustrated in FIG. 4. Locking mechanism 43 comprises a latching member 431 and a biasing member 432. Latching member 431 comprises a bar having a proximal end and a distal end having a latching face 433 configured to latch against a portion of body 2 when plunger 3 is in its retracted position, the proximal end being pivotably secured within a proximal portion of the plunger. Biasing member 432 is configured to bias the distal end of latching member 431 to extend laterally outwards of plunger 3 such that the latching face 433 of latching member 431 latches against the portion of body 2 when the plunger is in its retracted position, thereby preventing distal movement of plunger 3. Latching member 431 is positioned along the length of plunger 3 such that, when plunger 3 is positioned distally of its retracted position, latching member 431 is prevented from extending in a direction laterally outwards of plunger 3.

The proximal portion of plunger 3 may include any part of the plunger which is proximal of the lateral plane in which latching member 431 latches against a portion of body 2 when the plunger is locked in its retracted position.

The portion of body 2 which latching face 433 latches against may be a portion of the most distal end of the body. In an alternative embodiment, body 2 may include at least one flange formed at its proximal end, against which the latching member acts. The at least one flange may also provide a finger support surface for supporting fingers of a user operating the syringe when moving the plunger distally from its retracted position. In some embodiments, body 2 may include an opening at a location corresponding to the location of the distal end of latching member 431, which extends laterally outwards of the interior of body 2 when plunger 3 is in its retracted position. The opening formed in body 2 is sized and shaped to receive the distal end of latching member 431, and may surround the distal end of the latching member when the plunger is in its retracted position. In this embodiment, the distal end of latching member 431 may further include a proximally facing barb positioned to about a proximal side of the opening in body 2, such that the locking mechanism 430 may prevent both distal and proximal movement of the plunger from its retracted position, and may therefore act as the automatic retraction stop mechanism 24. In an alternative embodiment, the opening may comprise a notch formed in the distal end of body 2.

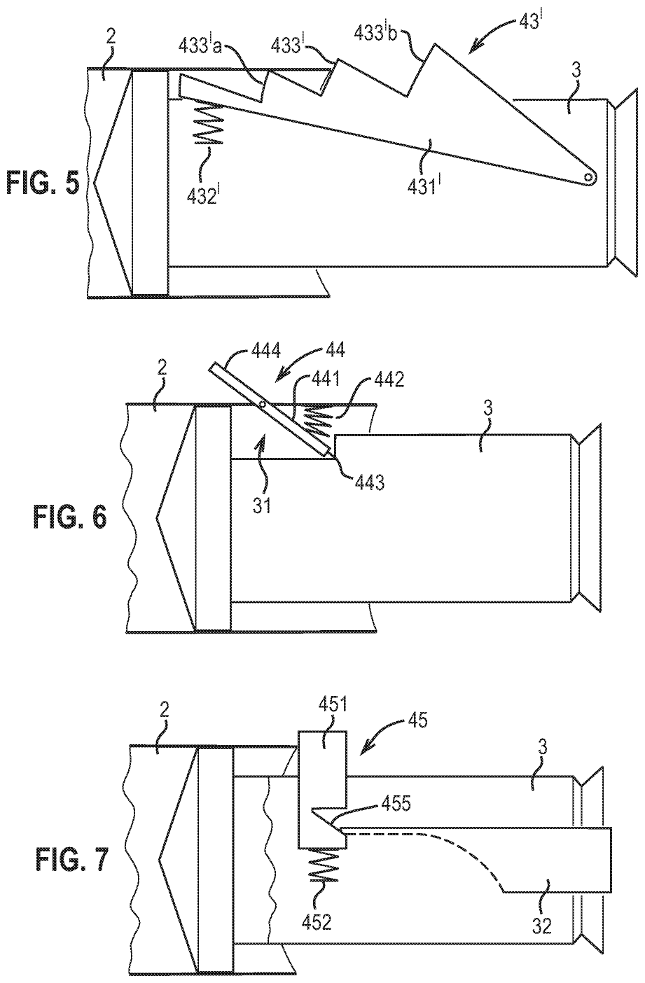

In some embodiments, multiple retracted positions of plunger 3 may be defined, which correspond to multiple volumes of cavity volume 21. Locking mechanism 43′ comprises a latching member 431′ and a biasing member 432′, and is shown in FIG. 5. In this embodiment, latching member 431′ may include more than one latching face 433′, 433′a, 433′b configured to latch against a portion of body 2 when the plunger is in its retracted position, these latching faces being positioned at locations corresponding to the portion of body 2 against which the latching member 431′ latches when the plunger is in each of its retracted positions respectively.

Latching member 431 may include additional features, such as tabs, which limit the extent to which latching member 431 can extend laterally outwards of plunger 3. Alternatively, the extent to which the latching member can extend laterally outwards of the plunger may be limited by biasing member 432. Biasing member 432 is illustrated to be a compression spring, although alternative biasing members are envisioned. For example, as shown in FIG. 4, biasing member 432 may additionally or alternatively comprise an elasticated band 432a, secured within a proximal portion of the plunger, and configured to bias latching member 431 about its pivot within plunger 3 such that the distal end of latching member 431 extends laterally outwards of the plunger. Biasing members 432 and 432a are illustrated together for ease of reference, and are not necessarily intended to be used together in the present invention. Use of both biasing spring 432 and biasing band 432a may, however, be used together to increase resistance to depression of the latching member in order to hinder accidental or unintentional unlocking of the plunger from its retracted position.

Locking mechanism 431 may be disengaged to allow for distal movement of plunger 3. A portion of the distal end of latching member 431 may be configured as a switch. In the illustrated embodiment, this portion is the surface of the latching member at approximately 90 degrees to latching face 433 and which faces outwards of the plunger, but may be any other suitable portion. To disengage the locking mechanism, a user may depress the switch portion of latching member 431, causing latching member 431 to retract in a direction laterally toward plunger 3, to a position in which the latching face does not latch against body 2 to prevent distal movement of the plunger.

In an alternative embodiment, as shown in FIG. 6, the biasing member 442 and latching member 441 of locking mechanism 44 may be located substantially in body 2. In this embodiment, the latching member 441 comprises a bar having a distal end and a proximal end having a latching face 443 configured to latch against a portion of plunger 3 when the plunger is in its retracted position, the bar being pivotably secured at a point distal of the latching face to a portion of body 2. Biasing member 442 is configured to bias the proximal end of the latching member bar to extend laterally inwards of the interior of the body, such that latching face 443 of latching member 441 latches against a portion of plunger 3, preventing proximal movement of the plunger. Latching member 441 is positioned along the length of the body such that, when the plunger is positioned distally of its retracted position, the proximal end of the latching member is prevented from extending laterally inwards of the interior of the body and latching against the plunger. This may be achieved by plunger 3 being substantially cylindrical along its length and including an opening or recessed portion 31 at a location corresponding to the location of the proximal end of the latching member when the plunger is in its retracted position (or multiple openings corresponding to the locations of the proximal end of the latching member when the plunger is in each of its retracted positions respectively). In this embodiment, when plunger 3 is in its retracted position and the proximal end of the latching member bar is biased laterally inwards of the interior of the body, a portion of the latching member bar which is distal of the pivot point may extend in a direction laterally outwards of body 2, to provide a lever 444 by which a user may disengage locking mechanism 44 to unlock the plunger from its retracted position.

### Single Use Feature

In some embodiments, body 2 or plunger 3 may further comprise a recessed portion sized and positioned to receive a portion of the latching member when the plunger is in its depressed position. As the biasing member of many of the locking mechanisms described herein remains active, if unsuccessful, in urging the latching member towards its engaged position following disengagement of the locking mechanism, when plunger 3 reaches its depressed position the latching member automatically extends into the recessed portion of the body or plunger, and latches against the sides of the recessed portion. In this configuration, the latching member cannot be accessed directly by a user (it is shielded by the body), hence the locking mechanism cannot be disengaged and the plunger is permanently locked in its depressed position. This prevents a used syringe from being reused.

### Actuation by Depressible Button

In further embodiments, plunger 3 comprises a button 32 movable between an actuated position and a non-actuated position, the button being configured to disengage the latching member from the portion of the body of the syringe when in its actuated position, thereby unlocking the plunger from its retracted position. In the specific examples of the button which are described herein, the button is located at the proximal end of the plunger, and actuation of the button is considered to comprise distal translation of the button with respect to the plunger. However, the button may be located at any suitable position on the syringe, and actuated by any suitable mechanism.

The latching member may comprise a camming surface, against which the button, when actuated, is configured to act in order to move the latching member in a direction laterally inwards of the plunger. The locking mechanisms may be similar in construction to those described above (and shown in FIGS. 2 to 6). Exemplary configurations of the interactions of the button and camming surfaces of the latching members are shown in FIGS. 7 and 8.

In the locking mechanism of FIG. 7, locking mechanism 45 comprises a latching member 451 and a biasing member 452, and is similar in construction to locking mechanism 41. Latching member 451 further comprises an inclined camming surface 455 upon which the button 32 is configured to act, when the button is actuated, to drive the latching member in a direction laterally towards plunger 3. Camming surface 455 has a length, and direction and degree of incline sufficient that, when the button reaches its actuated position, latching member 451 is positioned laterally inward of body 2, and plunger 3 is unlocked from its retracted position.

In the locking mechanism of FIG. 8, locking mechanism 46 comprises a latching member 461 and a biasing member 462, and is similar in construction to locking mechanism 43. A surface of the bar of latching member 461 acts as a camming surface upon which button 32 is configured to act, when the button is actuated, to drive latching member 461 laterally inwards such that, when the button reaches its actuated position, the distal end of latching member 461 is positioned laterally inward of body 2, and plunger 3 is unlocked from its retracted position.

The button of embodiments of the present invention may be configured in various ways to act, when actuated, upon the camming surfaces of the latching members. For example, the button may extend distally within the plunger as a sleeve or a rod which abuts the camming surface, or the button may engage an interim part which, in turn, abuts the camming surface. In FIGS. 7 to 9, the button extends distally within the plunger. The dashed line represents that a portion of the distally extending portion of the button has been cutaway for illustrative purposes.

In an alternative embodiment, shown in FIG. 9, locking mechanism 47 comprises a latching member 471 and a biasing member 472. Latching member 471 of locking mechanism 47 comprises teeth 475 and plunger 3 further comprises a gear wheel 35 configured to engage teeth 475. Actuation of button 32 is configured to cause rotation of the gear wheel in a direction which drives the latching member to retract in a direction laterally inwards of the plunger. For example, button 32 may extend on a rod or sleeve which includes teeth 36 configured to engage gear wheel 35.

Button 32 may, when plunger 3 is in its initial position, be provided in its actuated or non-actuated positions. When the button is provided in its actuated position when the plunger is in its initial position, the button may be driven to its non-actuated position upon force from the biasing member when the plunger is withdrawn to its retracted position. The button reaching its non-actuated position may provide an indication to a user that the plunger has been retracted sufficiently, and may be accompanied by at least one of an audible or tactile indicator.

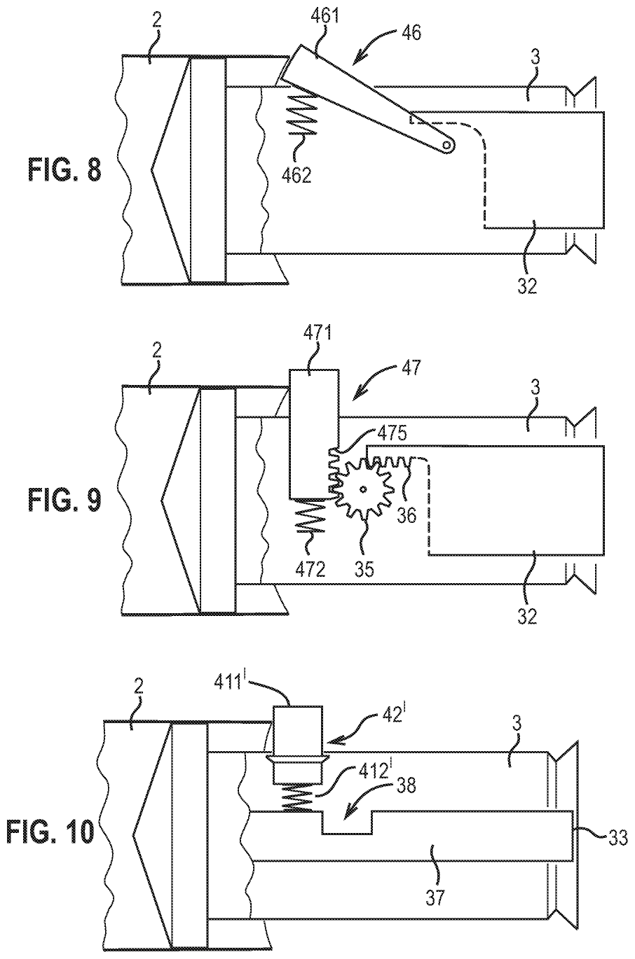

In an alternative embodiment, an example of which is illustrated in FIG. 10, plunger 3 further comprises an interior member 37 which extends distally from button 33. The interior member is configured to move longitudinally through the plunger concurrently with the button, and may be abutted by, or constituent of, button 33. Locking member 42′ may be similar in construction to locking member 41. Interior member 37 is positioned to be abutted by an end of biasing member 412′ when button 33 is in its non-actuated position, thereby providing support for the biasing member to urge latching member 411′ in a direction laterally outwards of the plunger to latch against a portion of body 2. Interior member 37 further comprises an opening 38 located at a position which corresponds to that of biasing member 412′ when button 33 is at its actuated position. Opening 38 is sized and shaped to allow, when button 33 is actuated, at least a portion of biasing member 412′ to translate into the opening, causing latching member 411′ to translate in a direction laterally inwards of the plunger to a point that latching member 411′ is disengaged from body 2, and plunger 3 is unlocked from its retracted position.

Translation of biasing member 412′, and therefore latching member 411′, into opening 38 may be under the influence of gravity. Alternatively, locking mechanism 42′ may comprise a contra-biasing member, such as an elastic band coupled to the latching member and/or the biasing member, configured to urge latching member in a direction laterally inwards of plunger 3. The contra-biasing member may take any suitable configuration, and will not bias sufficiently to overcome the biasing of biasing member 412′ in urging latching member 411′ in a direction laterally outwards of the plunger when button 33 is in its non-actuated position.

Opening 38 may extend through interior member 37 along the line of biasing by biasing member 412′ such that, when button 33 is actuated to disengage the locking mechanism, at least a portion of biasing member 412′ translates into the opening, causing latching member 411′ to translate in a direction laterally inwards of the plunger. Alternatively, opening 38 may comprise a recession in the interior member, as shown in FIG. 10.

According to embodiments, locking mechanism 42′ and its interaction with interior member 37 may serve to prevent unwanted reuse of the syringe, as opening 38 may be configured such that, once biasing member 412′ has translated therein, button 33 cannot be returned to its non-actuated position and biasing member 412′ cannot, therefore, bias latching member 411′ to extend laterally outwards of plunger 3 and to latch against a portion of the body of the syringe. This can prevent a user from accidentally reusing a used syringe by the methods described herein.

In embodiments of the syringes which include buttons configured to disengage the locking mechanisms, the locking mechanisms may be disengaged to unlock the plunger from its retracted position only when caused to do so by actuation of the buttons. Alternatively, the latching members may be configured to extend laterally outward of the plunger sufficiently that, when the locking mechanism is engaged, a user may also disengage the locking mechanism by depressing the latching member directly, causing it to retract in a direction laterally inward of plunger 3 to a position in which the latching member does not latch against body 2 to prevent distal movement of the plunger.

### Actuation by Rotatable Knob

In an alternative embodiment, plunger 3 comprises a knob, rotatable between an actuated and a non-actuated position, the knob being configured to disengage the latching member from the portion of the body of the syringe when in its actuated position, thereby unlocking the plunger from its retracted position. In the specific examples of the knob which are described herein, the knob is located at the proximal end of the plunger. However, the knob may be located at any suitable position on the syringe.

In some embodiments, the locking mechanism may be similar in construction to those described above (and shown in FIGS. 2 to 6), and the latching member may comprise a camming surface, against which the knob, when actuated, is configured to act in order to move the latching member in a direction laterally inwards of the plunger.

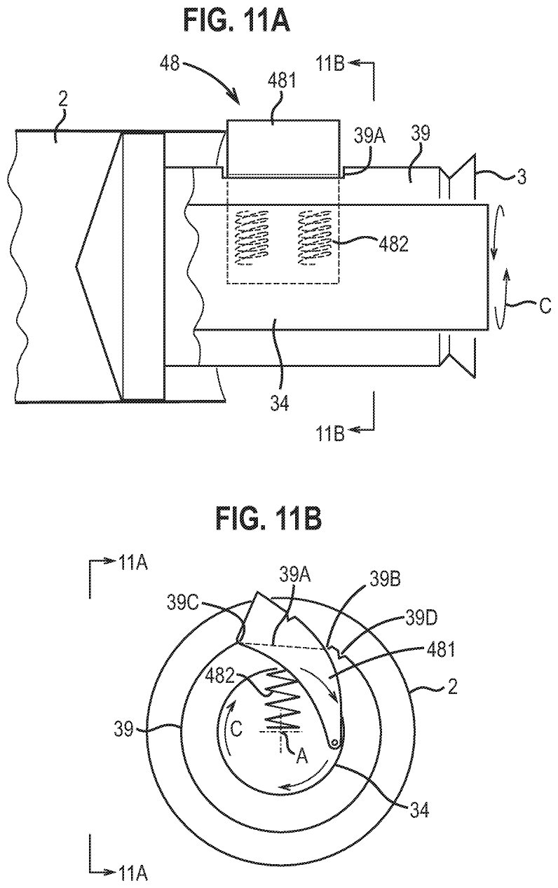

In an alternative embodiment, shown in FIGS. 11A and 11B, plunger 3 comprises a cylindrical body portion 39 comprising an opening 39A through which the latching member 481 of locking mechanism 48 is configured to extend laterally when the plunger is withdrawn to its retracted position and upon force from biasing member 482. The latching member may be pivotably secured, along an axis parallel to the longitudinal axis A of the syringe, to the knob 34 and interior to the cylindrical body portion 39 of the plunger, such that rotation of the knob causes rotation of the latching member relative to the cylindrical body portion of the plunger. The biasing member is also configured to rotate concurrently with the knob, and relative to the cylindrical body portion of the plunger.

Viewed along the longitudinal axis A from the proximal end to the distal end, as shown in FIG. 11B, the opening 39A, which is shown using dashed lines, in the cylindrical body of the plunger has a first edge 39B and an second edge 39C. Latching member 481 may be pivotably secured to the knob 34 at one end, such that a first end of the latching member is retained within the cylindrical body 39 of the plunger.

The biasing member 482 is configured to bias a second end of the latching member to extend laterally outwards of the plunger, through the opening 39A in the cylindrical body of the plunger, such that a portion of the latching member latches against a portion of body 2 when the plunger is in its retracted position, thereby preventing distal movement of plunger 3.

The second end of the latching member is configured to abut the second edge 39C of the opening in the cylindrical body of the plunger such that, when the plunger is locked in its retracted position, neither latching member 481 nor knob 34 can be rotated in a direction opposite to direction C.

To unlock the plunger from its retracted position, knob 34 is rotated in a direction C. Rotation of the knob in direction C causes corresponding rotation of the latching member 481 in direction C, with respect to the cylindrical body 39 of the plunger. As the latching member is rotated, the surface of the latching member which faces approximately outwards of the plunger engages the first edge 39B of the opening in the cylindrical body of the plunger, such that the latching member is progressively, from its first end toward its second end, translated interiorly of the cylindrical body of the plunger. The latching member is sufficiently rigid that progressive translation of the latching member into the cylindrical body of the plunger causes depression of the second end of the latching member in direction laterally inwards of the plunger. The cylindrical body of the plunger may further comprise a lip 39D adjacent the first edge 39B of the opening, configured to retain the second end of latching member 481 once the knob has been rotated sufficiently for the plunger to be unlocked from its retracted position.

In the embodiment shown in FIG. 11B, direction C is clockwise. In alternative embodiments, the locking mechanism may be configured such that anticlockwise rotation of the knob causes depression of the latching member in a direction laterally inwards of the plunger and, therefore, allows the plunger to be unlocked from its retracted position.

### Flexible Seal

As described in the “structure” section above, at least a portion of plunger 3 is configured to create an air tight seal with the interior of the syringe body 2, such that a decreased pressure is created within cavity volume 21 when plunger 3 is moved proximally. The portion of plunger 3 which is configured to create an air tight seal with the interior of body 2 may comprise a sealing member. The sealing member may comprise any suitable feature for sealing against the internal surface of the body, to prevent air and/or fluid flow between the cavity volume 21 and an external environment. One such sealing member will now be described in greater detail, with reference to FIGS. 13A, 13B, and 14.

Syringe 10 comprises a body 200, and a plunger 300 which is moveable between an initial position, shown in FIG. 13A, and a retracted position, shown in FIG. 13B. As in syringe 1, body 200 and plunger 300 define a cavity volume 21 within the syringe. Syringe 10 has a generally cylindrical shape. Body 200 has a generally cylindrical shape. The distal end of body 200 may include a frustoconical shape (or substantially/approximately frustoconical shape), terminating at its distal end with a discharge opening 22, as described previously. The proximal end of body 200 may comprise one or more finger support surfaces for supporting one or more of a user's digits when the user is moving the plunger distally from its retracted position. The retracted position of plunger 300 may be defined by the position of the plunger at which a specific and desired volume of the cavity volume is defined by the body and plunger.

Plunger 300 is shaped and sized to fit within the interior of body 200. The plunger comprises a piston portion, and a flexible seal 310 configured to create an air tight seal with the interior of the syringe body 200. The flexible seal 310 is coupled to a proximal region of the body 200 of the syringe, and expands over at least the distal end of the piston as the plunger 300 is moved distally. The flexible seal may be considered to have an expanded configuration (FIG. 13A) and a contracted configuration (FIG. 13B). It can be appreciated that the flexible seal 310 is capable of expanding to allow for full depression of the plunger 300 within the body 200, without tearing or becoming detached from the body. As the plunger 300 is retracted within the body, the flexible seal is biased to automatically contract towards its contracted configuration, due to, for example, the resilience of the flexible seal 310 or an additional resilient member connected thereto. The flexible seal provides for a chamber seal, i.e. the flexible seal prevents air and fluid flow between the cavity volume 21 and an external environment. Also, the flexible seal provides for the maintenance of a sterile pathway within the cavity volume. It can be appreciated that, when a plunger of some conventional syringes is retracted, a portion of the body interior which was previously exposed to an external environment then defines part of the internal cavity volume. This can be detrimental in allowing contamination of the syringe interior. With the flexible seal 310, the interior of the body 200 is not exposed to an external environment, regardless of plunger 300 position.

In some embodiments, the flexible seal 310 is configured to stretch from the contracted configuration to the expanded configuration, and to relax from the expanded configuration to the contracted configuration. In other words, in these embodiments the expanded configuration is a stretched configuration and the contracted configuration is a relaxed configuration. In some embodiments, the flexible seal is biased to compress, collapse or otherwise transition towards its contracted or relaxed configuration.

Syringe 10′, illustrated by FIG. 14, comprises a body 200′, and a plunger 300′. Syringe 10 is identical to syringe 10 in all aspects except the shape of the internal surface of the distal end of the body 200′ and the shape of the distal end of the plunger 300′. The internal surface of the body 200′ at its distal end has a rounded shape, terminating at its very distal end with a discharge opening 22, as described previously. The distal end of the plunger 300′ is correspondingly rounded. The rounded portions may include an approximately frustoconical shape, with a rounded tip and rounded edges in the region that the distal end transitions to the approximately cylindrical shape of the more proximal portion of the syringe components. In an embodiment, the internal surface of the body at its distal end and the distal end of the plunger are hemi-ellipsoidal. In other embodiments, the internal surface of the body at its distal end and the distal end of the plunger are hemispherical.

The rounded configuration of this embodiment is particularly beneficial in, but is not limited to, syringes comprising a flexible seal 310. The rounded shape of the piston of the plunger presents fewer, or no, concentrated points of strain on the flexible seal being stretched or expanded thereover. This beneficially reduces the likelihood of tearing of the flexible seal during use, and enables the use of thinner, or less strong materials for the flexible seal.

The flexible seal 310 may be coupled to the body 200, 200′ by any suitable means. For example, the flexible seal 310 may be clamped or screwed to or within a portion of the body 200, 200′, or affixed using adhesive or via heat treatment. Alternatively, the flexible seal 310 may comprise a slot around its circumference which can fixedly engage a ridge or flange formed on the body 200, 200′. Such a ridge or flange may be formed, for example, on an external or proximal surface of the body, or on finger support surfaces formed in or on the body. In an embodiment, the flexible seal may be coupled within a space formed within finger support surfaces formed in or on the body. The proximal region of the body to which the seal is coupled need not necessarily be the most proximal region of the body, but rather any position which allows for retraction of the plunger 300, 300′ to its retracted position.

In an embodiment, the entire circumference of the flexible seal 310 may be coupled to the body 200, 200′. In an alternative embodiment, only portions of the flexible seal 310 need be coupled to the body. For example, the flexible seal may be coupled to the body at one or more points or sections along the circumferential portion of the flexible seal.

The flexible seal 310 stretches or expands as the plunger 300, 300′ is moved distally. According to embodiments, the flexible sea 310 generally conforms to the shape of the piston of the plunger. As the plunger is moved proximally, the resilience of the flexible seal means that conformity between the flexible seal and the piston of the plunger is maintained. In embodiments, when the plunger is positioned in its depressed position the flexible seal also conforms to the shape of an internal surface of the body 200, 200′ at its distal end. Accordingly, this promotes maximal evacuation of the cavity volume when the plunger is fully depressed and, therefore, ensures that an entire dose is delivered.

In embodiments in which the flexible seal 310 is provided in a syringe in combination with a locking mechanism 4 or a retraction stop mechanism 24, it will be appreciated that coupling of the flexible seal to the body 200, 200′ should be at a position such that the flexible seal does not prevent the locking mechanism from operating effectively. For example, if the flexible seal is provided in a device in combination with one of the locking mechanisms shown in FIGS. 2, 4, 5, 7 to 11, the flexible 310 seal may be coupled to a portion of the body 200, 200′ which is distal of the portion of body against which the latching member latches when the locking mechanism engages (as the plunger 300, 300′ reaches its retracted position). Similarly, if the flexible seal is provided in a device in combination with one of the locking mechanisms shown in FIGS. 3 and 6, the flexible seal may be coupled to a portion of the body which is distal of the portion of body from which the latching member extends when the locking mechanism engages. A person of ordinary skill in the art will appreciate based on this disclosure that the flexible seal described above may be incorporated into different types of syringe having various other features, in a manner which does not impair the effectiveness of those other features.

The flexible seal 310 may have a neutral configuration (i.e. the configuration in which it is most relaxed, storing the least potential energy) into which it is automatically biased by the resilience of the flexible seal. This biasing of the flexible seal 310 to its neutral configuration may be advantageously utilised in syringes of these embodiments.

According to embodiments, the flexible seal 310 may be arranged, for example by being coupled to the body at a neutral position, to assume its neutral configuration when the plunger 300, 300′ is in a position which defines a specific desired volume of the cavity volume for normal use of the syringe. This configuration can help to ensure that the correct volume of fluid is drawn into the cavity volume to fill the specific desired volume. The resistive force resulting from the resilience of the flexible seal when in its non-neutral configuration will help to create and maintain a decreased pressure within the cavity volume as fluid is drawn therein. The resilience of the flexible seal may be sufficient to ensure that a predetermined amount of fluid must be drawn into the cavity volume for equilibrium between the pressure of the cavity volume and ambient pressure to be reached.

Alternatively, the flexible seal 310 may be arranged to assume its neutral configuration when the plunger 300, 300′ is distal of its retracted position. In such configurations, the resistive force resulting from the resilience of the flexible seal when in its non-neutral configuration may help to initiate proximal movement of the plunger from its retracted position, once the plunger is free to move. This initiation may be advantageous to users with reduced or impaired motor abilities.

In certain embodiments, the flexible seal 310 may be arranged to assume its neutral configuration when the plunger 300, 300′ is in its depressed configuration (i.e. the neutral configuration of the flexible seal is the same as its “expanded” or “stretched” configuration as used in the present context). In such configurations, the resistive force resulting from the resilience of the flexible seal when in its non-neutral configuration (e.g. its “contracted” or “relaxed” configuration as used in the present context) may cause automatic depression of the plunger from its retracted position towards its depressed position and, therefore, automatic expulsion of the fluid contained in the cavity volume, once the plunger is free to move.

In embodiments in which the flexible seal is configured to assume its neutral configuration when the plunger is in any position other than its retracted position, and when this configuration is combined in a syringe with a locking mechanism 4 such as those described herein, the resistive force or potential provided by the flexible seal when the plunger is in its retracted position should be less than the retaining force provided by the locking mechanism.

The flexible seal 310 may be provided to overlay the piston of the plunger 300, 300′ without any permanent or temporary fixation therebetween. The piston and flexible seal in such embodiments may engage due to their relative positions in the body. Alternatively, a central portion of the flexible seal 310 can be coupled to a corresponding portion of the distal end of the piston. For example, the central portion of the flexible seal may be fixed to a corresponding area of the piston's distal end by any suitable adhesive or by heat treatment. In some embodiments, a central portion 310A of the flexible seal may be configured to reside within a bore or socket 320 formed within the piston. In an embodiment, the central portion 310A of the flexible seal may include a plug, sized and positioned to fit within the corresponding bore or socket 320 formed within the piston. The plug may be temporarily or permanently fixed within the bore or socket 320 by, for example but not limited to, friction, interlocking features, or adhesive. This fixation of the central portion of the flexible seal to a corresponding portion of the plunger's distal end can help to ensure approximately symmetrical transitioning, stretching, expansion, compression, contraction, etc. of the flexible seal around the distal end of the plunger as the plunger moves proximally and/or distally, between its retracted, initial, and depressed positions.

The flexible seal 310 may comprise any suitable materials for expanding or contracting as outlined above. For example, the flexible seal may comprise a medical grade rubber, or elastomer which exhibits rubber like physical properties. The flexible seal 310 may be provided such that the thickness of a side portion of the flexible seal which is overlaid along a side of the piston when the plunger 300, 300′ is in its depressed position is in the range of 1mm to 5mm. The flexible seal 310 may be provided such that the thickness of the portion of the flexible seal which overlays the piston when the plunger 300, 300′ is in its retracted position is in the range of 1mm to 8mm. The materials used may be selected to allow for a desired flexibility, a desired potential energy to be stored in the flexible seal when stretched to various degrees, for maximal strength, for their fluid impermeability, or for other reasons.

In some embodiments, the volume of the bore or socket 320 formed in the piston may be greater than the volume of the central portion 310A of the flexible seal which is affixed within. The central portion 310A of the flexible seal may be held within the bore or socket 320 by a resilient member, which biases at least some of the flexible seal into the bore or socket 320. When the plunger is retracted, the flexible seal is biased to collect into the bore or socket, such that portion of the flexible seal which remains outside of the bore or socket approximately conforms to the piston end. When plunger 300, 300′ is depressed, more of the flexible seal is external to the bore or socket 320, as it has expanded out of the bore or socket to accommodate the greater coverage of the piston required when the piston is moved distally within the syringe body. The central portion 320A of the flexible seal remains within or adjacent to the bore or socket, as it is held by the resilient member.

Additionally or alternatively, the flexible seal may be biased to retract within a portion of the body 200, 200′ of the syringes when the plunger is moved proximally toward its retracted position.

As described in relation to syringe 1, syringes 10 and 10′ may comprise an automatic retraction stop mechanism 24, configured to prevent plunger 300, 300′ from being moveable to a position proximal of its retracted position, or to allow plunger 300, 300′ to be moveable to a position proximal of its retracted position, but prevent the plunger from being moveable proximal of a maximum capacity position. One or both of body 200, 200′ and plunger 300, 300′ may include markings thereon to denote the volume or capacity of the cavity volume defined by the body and plunger at a given position of the plunger. The markings may provide a user with an indication and/or confirmation that the plunger has reached, or is approaching, its retracted position. The markings may also be used to verify that the correct syringe is being used for the required dosage.

The sealing member 310 and syringe embodiments 10, 10′ illustrated by FIGS. 13 and 14 may be incorporated into devices alongside any of the other features described herein (such as, but not limited to, the various locking mechanisms 4, the retraction stop mechanism 24, the single-use features, and/or the various actuation mechanisms), and may be utilised in any of the methods described herein.

Replaceable End Cap with Integrated Flexible Seal

In some embodiments, the flexible seal 310 is coupled to a proximal region of the body 200, 200′ of the syringe via an end cap 50. The end cap may be configured for removable attachment to the proximal end of the body. For example, a distal portion of the end cap may be formed with a screw, clip, snap fit or locking fit connection configured to engage a corresponding feature formed on a proximal region of the body 200, 200′. Alternatively, the end cap may be configured for a fixed attachment to the body, for example by adhesive or compression fit.

The flexible seal 310 may be coupled to or within the end cap by any suitable means. Alternatively, the flexible seal 310 may be held between the end cap 50 and the body 200, 200′, for example via compression fit, when the end cap is attached to the proximal end of the body.

The end cap 50 has an aperture 52 therethrough having a cross-section suitable to moveably receive a piston portion of the plunger 300, 300′, such that the flexible seal stretches or expands over at least the distal end of the piston as the plunger 300, 300′ is moved distally through the aperture 52.

The end cap may comprise a latch receiving component 51 (e.g. an aperture or channel) for receiving the latching member of locking mechanism 4 when the end cap is in place on the syringe body and the plunger is in its retracted position, thereby preventing proximal movement of the plunger. Alternatively, the end cap may comprise a latching member configured to extend laterally inwards of the interior of the end cap, such that a portion of the latching member latches against a portion of plunger (e.g. opening or recessed portion 31) when the plunger is in its retracted position, thereby preventing proximal movement of the plunger. In some embodiments, the end cap 50 may further comprise the automatic retraction stop mechanism 24, configured to prevent the plunger from being moveable to a position proximal of its retracted position or maximum capacity position.

The end cap with integrated flexible seal provides a number of benefits. For example, the end cap may allow for the use of a single-use flexible seal with a multiple-use syringe body and/or plunger, or vice-versa. The end cap may allow for the various components of the syringe to be sterilised or reconditioned separately. This may be particularly beneficial if, for example, the optimal methods or substances for sterilising or reconditioning the syringe body are different to those, or unsuitable, for use on the flexible seal. The end cap may also be configured for attachment to a standard syringe body, to provide the flexible seal 310 and/or locking mechanisms 4 and/or retraction stop mechanism 24, and the benefits associated with these features.

### Method of Reconstituting a Pharmaceutical Product

As described earlier in this application, the embodiment described herein can be used in methods for preparing a syringe of a mixed pharmaceutical product. Conventional methods typically involve a large number of steps where connections are made between vials, needles and syringes. These methods involve the provision of two or more components of a product in separate containers, using a syringe to transfer one component into the container of the other component for mixing, and using a further syringe to draw in the mixed product. These conventional methods involve many steps, present many opportunities for needle stick injuries and contamination, and require several pieces of apparatus.

According to an aspect of the present invention is a method 1200 for preparing a syringe of a mixed pharmaceutical product. Preparation is carried out by providing a syringe, such as a syringe described in this application. The syringe is provided, in step 1202, with the plunger in its initial position, and with a first component of the mixed pharmaceutical product 101 contained in the defined cavity volume, as shown in FIG. 1A. Current methods of reconstitution of a mixed pharmaceutical product require multiple syringes and separate containers of the product's components to be stored prior to mixing. Providing the first component in the syringe therefore reduces the amount of space required for storage. The first component may be provided in syringes by the manufacturer, or may be loaded into syringes at a time prior to administration by a user. Provision of the first component of the mixed pharmaceutical product in the syringe also removes the requirement of a user to accurately measure out an appropriate dose.

At step 1204, the discharge opening 22, and therefore the cavity volume 21, is then placed into fluid communication with a vial 100 containing a second component of the mixed pharmaceutical product 102. This step may involve uncapping, or otherwise opening of a fluid path through discharge opening 22, and may further involve attaching a needle or transfer member to the discharge opening. If the vial 100 containing the second component of the mixed pharmaceutical product is sealed by a protective seal, this step may also involve removing the protective seal. Alternatively, the discharge opening, needle or transfer member may be used to pierce the protective seal in order to create a fluid path between cavity volume 21 and vial 100. In such an embodiment, the tip of the piercing member may be provided with a slanted profile that arrives at a point to aid penetration and to avoid needle coring of the protective seal. The needle, transfer member, or discharge opening of the syringe must, at this stage, extend into vial 100 sufficiently for at least the syringe tip (i.e. the distal end of the needle/transfer member/discharge opening) to be submerged in the second component of the mixed pharmaceutical product.

At step 1206, while the tip of the needle, transfer member, or discharge opening 22 remains submerged in the second component of the mixed pharmaceutical product, and the cavity volume is, therefore, in fluid communication with the vial containing the second component of the mixed pharmaceutical product, plunger 3 is retracted distally to its retracted position, as shown in FIG. 1B. This retraction increases the volume of cavity volume 21, thereby causing a decreased pressure, with respect to both ambient pressure and pressure in the cavity volume prior to retraction of the plunger, within the cavity volume to be created. Under the influence of the decreased pressure, the second component of the mixed pharmaceutical product 102 begins to be drawn into the cavity volume via the fluid path created through discharge opening 22. Typically, the vial is held upright during steps 1204 and 1206, with the syringe held above it with respect to gravity, as is shown in FIG. 1B. In this arrangement of syringe and vial, the user may position the syringe such that the syringe tip reaches the bottom of vial 100, and be assured that, as the second component of the mixed pharmaceutical product is drawn into the volume cavity, gravity will cause the portion of the second component which remains in the vial to move towards the bottom of the vial and, therefore, the syringe tip. Hence, the syringe tip remains continually submerged in the second component of the mixed pharmaceutical product while the volume cavity fills, and ingress of air into the syringe as the volume cavity 21 fills is avoided.

When plunger 3 reaches its retracted position at step 1208, locking mechanism 4 engages automatically to prevent proximal movement of the plunger. The locking mechanism may also prevent further distal motion of the plunger. While the plunger is locked in its retracted position at step 1210, the effects of the decreased pressure within the cavity volume upon the plunger are mitigated, and the second component of the drug continues to be drawn into the cavity volume until equilibrium between the pressure of the cavity volume and ambient pressure can be reached.

The retracted position of the plunger at which the locking member is configured to engage may correspond to a desired volume of total mixed pharmaceutical product or second component of the mixed pharmaceutical product being present in the syringe, allowing for accurate preparation of a syringe containing a specific dose of mixed pharmaceutical product, or for preparation of a syringe containing a pharmaceutical product which has been mixed from components at specific ratios. This removes the requirement for a user to accurately measure the volumes of components in the syringe, making the process of preparing a syringe of a mixed pharmaceutical product faster, more consistent, and more accurate, even when completed by a user who is not a skilled health practitioner.

In some embodiments, once cavity volume 21 has been filled, discharge opening 22 may be removed from fluid communication with vial 100, as in step 1212. In further embodiments, fluid communication between the cavity volume and the exterior of the syringe may then be prevented by capping of the discharge opening, needle or transfer member.

Following the second component of mixed pharmaceutical 102 product having been drawn into the cavity volume and prior to administration of the mixed pharmaceutical product 103, the mixed pharmaceutical product is reconstituted by mixing of the first and second components at step 1214. In some embodiments, syringe 1 may be held stationary for a period of time which is sufficient for the first and second components to mix to form the mixed pharmaceutical product 103. In alternative embodiments, the syringe may be shaken or otherwise agitated to mix the components.

In some embodiments, at a time following the second component of mixed pharmaceutical product having been drawn into the cavity volume and prior to administration of the mixed pharmaceutical product, the needle or transfer member may be replaced by an administration needle. Provision of a different needle or transfer member for administration reduces the risk of contaminants from the vial, protective seal, or otherwise, being transferred to a patient. It also reduces the risk of traces of the second component of the mixed pharmaceutical product (which may reside in the first needle or transfer member when the components within the cavity volume are mixed to form the mixed pharmaceutical product) from being transferred.

Following preparation of the syringe with the mixed pharmaceutical product 103 and prior to administration of the mixed pharmaceutical product, plunger 3 may be unlocked from its retracted position, as in step 1216 and shown in FIG. 1C. Locking mechanism 4 may include a latching member and a biasing member, wherein the latching member is configured, when the locking mechanism is engaged and upon force from the biasing member, to extend laterally outwards of plunger 3 and to latch against a portion of body 2 of the syringe. The locking member is disengaged by depression of the latching member in a direction laterally toward the plunger. This depression may be initiated by force upon the latching member itself, or by actuation of a button, knob, latch, switch, lever or otherwise, which is movable between an actuated position and a non-actuated position and configured to disengage the latching member from the portion of the body of the syringe when actuated. The positioning of such a button, knob, etc. may be adapted according to the requirements of the intended user. Resistance to depression of the latching member or to actuation of the button, knob, etc. may be adapted to the requirements of the intended user, or in order to hinder unlocking of the plunger from its retracted position by those other than the intended user.

Following preparation of the syringe with the mixed pharmaceutical product and unlocking of the plunger from its retracted position, the plunger is depressed proximally to a depressed position. The depressed position, shown in FIG. 1D, is one in which the plunger is depressed distally relative to the initial position, to provide for a further decreased cavity volume relative to the cavity volume defined by the body and the plunger in its initial position. Depression of the plunger from its retracted position to its depressed position causes expulsion of the mixed pharmaceutical product from the cavity volume via the discharge opening, for example, for administration of the mixed pharmaceutical product to a patient.

Other locking mechanisms of syringes which are suitable for preparation of a syringe of a mixed pharmaceutical product by the methods disclosed herein are discussed in this application, along with the mechanisms by which they may engage and disengage to selectively allow movement of plunger 3 from its retracted position.

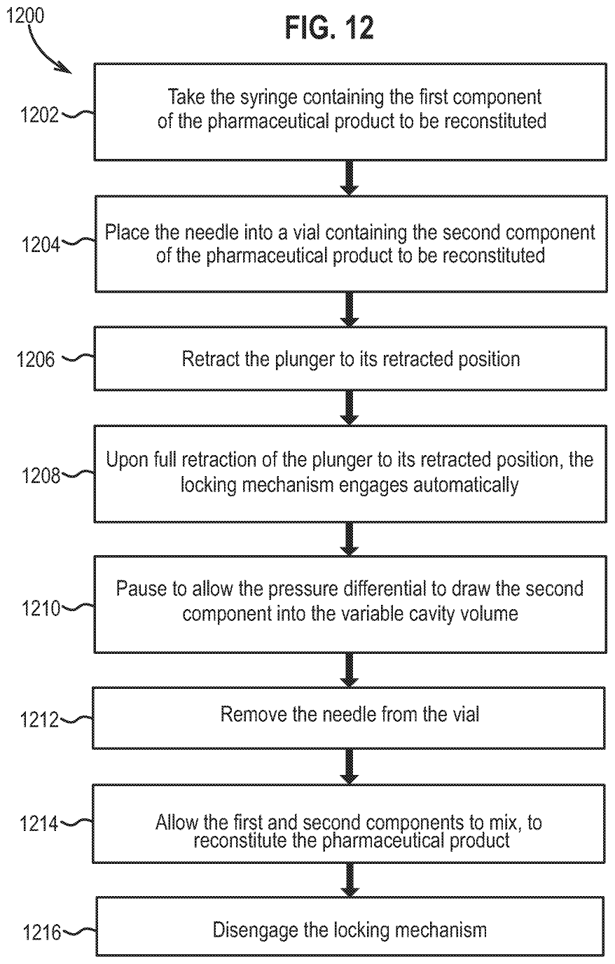

Unless specified, the steps of the method of the present invention are not limited to occur in the order in which they are described herein, or shown in FIG. 12. For example, the administration needle may be attached to the discharge opening before the syringe is agitated to mix the components at step 1214.

Additionally, a portion of the latching member, push switch, bar, button, or knob may be adapted to receive a digit of a user, making depression of the latching member, and therefore unlocking of the plunger from its retracted position, comfortable to the user. Additionally, these features may include a distinguishing indicia which identifies it to an inexperienced user. Exemplary indicia include, but are not limited to, a textured surface, a distinguishing color or pattern, or text.

Engaging and/or disengaging of the locking mechanism of the present invention may be accompanied by at least one of a visible, audible, or tactile indicator. For example, the latching member may include markings which are viewable through a window formed in plunger 3 when the latching member is depressed laterally toward the plunger sufficiently to unlock the plunger from its retracted position. Such an indication provides a user with confirmation of the state of the plunger, preventing them from, for example, damaging the syringe by attempting to move the plunger distally from its retracted position while the lock is engaged.

The mixed pharmaceutical product may be reconstituted Remicade (RTM) (INN: infliximab). The first component of the mixed pharmaceutical product may be Remicade (RTM) in the form of a lyophilized powder, a lyophilized cake, or lyophilized granules. The second component of the mixed pharmaceutical product may be sterilised water. In some embodiments, the components may be of the mixed pharmaceutical product Sylvant (RTM) (INN: siltuximab)

While the invention is described in relation to reconstitution of a mixed pharmaceutical product from two components, use of the disclosed devices and methods for the reconstitution or mixing of a pharmaceutical product from more than two components is envisaged. Use of the disclosed devices for the administration of single component pharmaceutical products is also envisaged.

It will be appreciated that the above disclosure provides specific examples of certain implementations of the invention, and that modifications can be made within the scope of the claims.

### NUMBERED EMBODIMENTS OF THE INVENTION

1. A syringe, comprising:
a body;
a plunger moveable between an initial position and a retracted position within and relative to the body; and
a locking mechanism configured to lock the plunger in the retracted position relative to the body,
wherein the body and plunger define a cavity volume within the syringe, wherein the capacity of the cavity volume is variable according to the position of the plunger within the body.

2. The syringe of embodiment 1, wherein the body comprises a discharge opening at a distal end and a plunger receiving opening at a proximal end.

3. The syringe of embodiment 1 or embodiment 2, wherein the retracted position is one in which the plunger is retracted proximally relative to the initial position, to provide for an increased cavity volume relative to the cavity volume defined by the body and the plunger in the initial position.

4. A syringe, comprising:
a body;
a plunger moveable between an initial position and a retracted position within and relative to the body, wherein the retracted position is one in which the plunger is retracted proximally relative to the initial position, to provide for an increased cavity volume relative to the cavity volume defined by the body and the plunger in the initial position; and
a locking mechanism configured to automatically engage when the plunger is withdrawn to the retracted position to lock the plunger in the retracted position relative to the body, to prevent at least distal movement of the plunger from the retracted position,
wherein the body and plunger define a cavity volume within the syringe, wherein the capacity of the cavity volume is variable according to the position of the plunger within the body.

5. The syringe of embodiment 4, wherein the body comprises a discharge opening at a distal end and a plunger receiving opening at a proximal end.

6. The syringe of any preceding embodiment, wherein the plunger is further moveable to a depressed position, wherein the depressed position is one in which the plunger is depressed distally relative to the initial position, to provide for a further decreased cavity volume relative to the cavity volume defined by the body and the plunger in the initial position.

7. The syringe of any preceding embodiment, further comprising an automatic retraction stop mechanism configured to prevent the plunger from being moveable to a position proximal of the retracted position.

8. The syringe of any preceding embodiment, wherein a portion of the plunger creates an air tight seal with the interior of the syringe body, such that a decreased pressure within the cavity volume is created within the cavity volume when the plunger is moved proximally. The portion of the plunger that creates an air tight seal with the interior of the syringe body may comprise rubber.

9. The syringe of any preceding embodiment, wherein the locking mechanism comprises a latching member extendable from the plunger to latch against a portion of the body of the syringe when the plunger is moved to the retracted position, thereby locking the plunger in the retracted position relative to the body.

10. The syringe of embodiment 9, the locking mechanism further comprising a biasing member for causing extension of the latching member, the latching member being configured, when the plunger is withdrawn to the retracted position and upon force from the biasing member, to extend laterally outwards of the plunger and to latch against the portion of the body of the syringe. In other words, the biasing member is adapted to extend the latching member, the latching member being configured to extend laterally outwards of the plunger and to latch against the portion of the body of the syringe when the plunger is withdrawn to the retracted position and upon exertion of force from the biasing member on the latching member.

11. The syringe of embodiment 9 or embodiment 10, wherein the portion of the body of the syringe against which the latching member is configured to latch when the plunger is in the retracted position is formed on a flange located at a proximal end of the body.

12. The syringe of embodiment 11, wherein the flange is a finger support surface for supporting fingers of a user operating the syringe when moving the plunger distally from the retracted position.

13. The syringe of embodiment 9 or embodiment 10, wherein the body comprises an opening through which the latching member is configured to extend when the plunger is withdrawn to the retracted position.

14. The syringe of any one of embodiments 9 to 13, wherein depression of the latching member laterally toward the plunger unlocks the plunger from the retracted position.

15. The syringe of any one of embodiments 1 to 8, wherein the locking mechanism comprises a latching member extendable from the body of the syringe to latch against a portion of the plunger when the plunger is in the retracted position, thereby locking the plunger in the retracted position relative to the body.

16. The syringe of embodiment 15, the locking mechanism further comprising a biasing member for causing extension of the latching member, the latching member being configured, when the plunger is withdrawn to the retracted position and upon force from the biasing member being exerted on it, to extend laterally inwards of the plunger and to latch against the portion of the plunger. In other words, the biasing member is adapted to extend the latching member, the latching member being configured to extend laterally inwards of the plunger and to latch against the portion of the plunger when the plunger is withdrawn to the retracted position and upon exertion of force from the biasing member on the latching member.

17. The syringe of embodiment 15 or embodiment 16, wherein the plunger comprises an opening through which the latching member is configured to extend when the plunger is withdrawn to the retracted position.

18. The syringe of any one of embodiments 15 to 17, wherein the body further includes a proximal end having at least one flange, and the latching member is extendable from within the at least one flange.

19. The syringe of embodiment 18, wherein the at least one flange is a finger support surface for supporting fingers of a user operating the syringe when moving the plunger distally from the retracted position,

20. The syringe of any one of embodiments 15 to 19, wherein depression of the latching member laterally outwards of the plunger unlocks the plunger from the retracted position.

21. The syringe of any one of embodiments 9 to 14, wherein the latching member comprises a bar having a proximal end and a distal end, the proximal end being pivotably secured within a proximal portion of the plunger, the distal end being configured to extend laterally outwards of the plunger and to latch against a portion of the body of the syringe.

22. The syringe of any one of embodiments 9 to 14 or embodiment 21, wherein the plunger further comprises a button movable between an actuated position and a non-actuated position, the button being configured to disengage the latching member from the portion of the body of the syringe when in the actuated position.

23. The syringe of embodiment 22, wherein the latching member comprises a camming surface and the button is configured, when actuated, to act against the camming surface to move the latching member in a direction laterally inwards of the plunger.

24. The syringe of embodiment 22 when dependent on any one of embodiments 9 to 14, wherein the latching member comprises teeth and the plunger further comprises a gear wheel configured to engage the teeth, wherein actuation of the button is configured to cause rotation of the gear wheel in a direction which drives the latching member to retract in a direction laterally inwards of the plunger.

25. The syringe of any one of embodiments 22 to 24, wherein the button is in the actuated position when the plunger is in the initial position and when the plunger is withdrawn to the retracted position, and upon force from the biasing member, the button is driven to the non-actuated position.

26. The syringe of embodiment 22, wherein the plunger further comprises an interior member which extends distally from the button and is positioned to be abutted by an end of the biasing member opposite to the latching member when the button is in the non-actuated position, the interior member being configured to move longitudinally through the plunger concurrently with the button, the interior member comprising an opening larger than the biasing member and located at a position which corresponds to that of the biasing member when the button is at the actuated position.

27. The syringe of any one of embodiments 9 to 14 wherein the plunger comprises a cylindrical body portion comprising an opening through which the latching member is configured to extend laterally when the plunger is withdrawn to the retracted position and upon force from the biasing member.

28. The syringe of any preceding embodiment, wherein one or both of the body and the plunger is configured, when the plunger is in the initial position or the depressed position, to prevent the latching member from extending laterally outwards of the plunger.

29. The syringe of any preceding embodiment, wherein the syringe is provided in an initial state with the plunger in the initial position, wherein the cavity volume of the syringe holds a first component of a pharmaceutical product. The pharmaceutical product may be a mixed pharmaceutical product, made up of at least two parts when reconstituted or mixed.

30. The syringe of embodiment 2, embodiment 3 when dependent on embodiment 2, embodiment 5 or of any one of embodiments 6 to 28 when dependent upon embodiment 2 or embodiment 4, wherein the syringe is provided in a second state with the plunger in the retracted position, wherein the cavity volume of the syringe holds a first component of a pharmaceutical product and a second component of the pharmaceutical product which has been drawn into the cavity volume via the discharge opening in response to retraction of the plunger from the initial position to the retracted position. The pharmaceutical product may be a mixed pharmaceutical product, made up of at least two parts when reconstituted or mixed

31. The syringe of embodiment 30, wherein the syringe is configured to expel the first component of the pharmaceutical product and the second component of the pharmaceutical product from the cavity volume via the discharge opening in response to depression of the plunger from the retracted position towards the depressed position.

32. The syringe of any one of embodiments 29 to 31, wherein the first component of the pharmaceutical product is in solid form.

33. The syringe of embodiment 32, wherein the first component of the pharmaceutical product is in the form of:
a lyophilized powder;
a lyophilized cake; or
lyophilized granules.

34. The syringe of any one of embodiments 29 to 33, wherein the second component of the pharmaceutical product is a liquid.

35. The syringe of any one of embodiments 29 to 34, wherein the pharmaceutical product is reconstituted Remicade®, or Sylvant®.

36. The syringe of embodiment 8, or any one of embodiments 9 to 35 when dependent upon embodiment 8, wherein the portion of the plunger that creates an air tight seal with the interior of the syringe body comprises a flexible seal coupled to a proximal region of the body of the syringe and having an expanded configuration and a contracted configuration, the flexible seal being configured to transition from the expanded configuration to the contracted configuration as the plunger is moved proximally, from the initial position towards the retracted position, and/or configured to expand from the contracted configuration to the expanded configuration as the plunger is moved distally, from the retracted position towards or past the initial position. The flexible seal may be configured to stretch from the contracted configuration to the expanded configuration, and relax from the expanded configuration to the contracted configuration.

37. The syringe of embodiment 36, wherein an entire circumference of the flexible seal is coupled to the body. The flexible seal may be coupled to a proximal end cap which is attached to the body at a proximal end of the body. The proximal end cap may be configured for removable attachment to the body, or for a fixed attachment to the body.

38. The syringe of embodiment 36 or embodiment 37, wherein the plunger further comprises a piston, and a central portion of the flexible seal is coupled to a distal end of the piston.

39. The syringe of any one of embodiments 36 to 38, wherein an internal surface of the body at the distal end is rounded, and a distal end of the plunger is rounded.

40. The syringe of any one of embodiments 36 to 38, wherein an internal surface of the body at the distal end is hemi-ellipsoidal, and a distal end of the plunger is hemi-ellipsoidal.

41. The syringe of embodiment 40, wherein an internal surface of the body at the distal end is hemispherical, and a distal end of the plunger is hemispherical.

42. The syringe of any one of embodiments 36 to 41, when dependent on any of embodiments 3 to 6, wherein the flexible seal conforms to the shape of the distal end of the piston when the plunger is positioned in or between the retracted position and the depressed position.

43. The syringe of embodiment 42, or any one of embodiments 36 to 41 when dependent on any of embodiments 3 to 6, wherein an expanded portion of the flexible seal conforms to the shape of an internal surface of the distal end of the body when the plunger is in the depressed position.

44. A method of reconstituting a pharmaceutical product, comprising;
providing a syringe having a body, a plunger moveable between an initial position and a retracted position relative to the body, a discharge opening, a locking mechanism configured to lock the plunger in the retracted position, and a cavity volume defined by the body and plunger, wherein the plunger is provided in the initial position, and the defined cavity volume contains a first component of the pharmaceutical product (in other words, the cavity volume defined by the body and the plunger in the initial position contains the first component of the mixed pharmaceutical product);
placing the discharge opening into fluid communication with a vial containing a second component of the pharmaceutical product;
retracting the plunger to commence drawing in of the second component of the pharmaceutical product into the cavity volume;
upon full retraction of the plunger, the locking mechanism engaging;
wherein the second component continues to be drawn in while the locking mechanism is engaged, until the cavity volume defined by the body and the plunger in the retracted position is filled or the drawing in of the second component of the pharmaceutical product into the cavity volume defined by the body and the plunger in the retracted position is terminated. The pharmaceutical product may be a mixed pharmaceutical product, made up of at least two parts when reconstituted or mixed

45. A method of reconstituting a pharmaceutical product, comprising:
providing a syringe having a body, a plunger moveable between an initial position and a retracted position relative to the body, a discharge opening, a locking mechanism configured to lock the plunger in the retracted position to prevent at least distal movement of the plunger from the retracted position, and a cavity volume defined by the body and plunger, wherein the plunger is provided in the initial position, and the defined cavity volume contains a first component of the pharmaceutical product (in other words, the cavity volume defined by the body and the plunger in the initial position contains the first component of the mixed pharmaceutical product);
placing the discharge opening into fluid communication with a vial containing a second component of the pharmaceutical product;
retracting the plunger to commence drawing in of the second component of the pharmaceutical product into the cavity volume;
upon full retraction of the plunger, the locking mechanism automatically engaging;
wherein the second component continues to be drawn in while the locking mechanism is engaged, until the cavity volume defined by the body and the plunger in the retracted position is filled or the drawing in of the second component of the pharmaceutical product into the cavity volume defined by the body and the plunger in the retracted position is terminated. The pharmaceutical product may be a mixed pharmaceutical product, made up of at least two parts when reconstituted or mixed

46. The method of embodiment 44 or embodiment 45, wherein the first component of the pharmaceutical product is in solid form.

47. The method of embodiment 46, wherein the first component of the pharmaceutical product is in the form of:
a lyophilized powder;
a lyophilized cake; or
lyophilized granules.

48. The method of any one of embodiments 44 to 47, wherein the second component of the pharmaceutical product is a liquid.

49. The method of any one of embodiments 44 to 48, wherein the pharmaceutical product is reconstituted Remicade® or Sylvent®.

50. The method of any one of embodiments 44 to 49, further comprising unlocking the locking mechanism after the second component of the pharmaceutical product has been drawn into the cavity volume defined by the body and the plunger in the retracted position.

51. The method of embodiment 50, wherein the locking mechanism comprises a latching member, and unlocking the locking mechanism comprises a user pushing the latching member toward the longitudinal axis of the syringe.

52. The method of embodiment 50 or embodiment 51, wherein the plunger comprises a button configured to engage the locking mechanism, and unlocking the locking mechanism comprises a user actuating the button.

53. The method of embodiment 51, or embodiment 52 when dependent on embodiment 51, wherein the locking member further comprises a biasing member for causing extension of the latching member, such that the latching member engages upon full retraction of the plunger. In other words, the biasing member is adapted to extend the latching member to engage the locking mechanism upon full retraction of the plunger.

54. The method of any one of embodiments 44 to 53, further comprising shaking the syringe after the second component of the pharmaceutical product has been drawn into the cavity volume.

55. The method of any one of embodiments 44 to 54, further comprising removing the discharge opening from fluid communication with the vial and replacing the discharge opening after the second component of the pharmaceutical product has been drawn into the cavity volume defined by the body and the plunger in the retracted position.

56. The method of any one of embodiments 44 to 55, wherein the syringe is the syringe of any of embodiments 1 to 14 or embodiments 21 to 43.

57. The method of embodiment 52, or any one of embodiments 53 to 55 when dependent upon embodiment 52, wherein the syringe is the syringe of any of embodiments 22 to 26, or the syringe of any one of embodiments 28 to 43 when dependent upon embodiment 22.

58. A system for reconstituting a pharmaceutical product, comprising:
a syringe comprising a body, a plunger moveable between an initial position and a retracted position relative to the body, a discharge opening, a locking mechanism configured to lock the plunger in the retracted position, and a cavity volume defined by the body and plunger, wherein the capacity of the cavity volume is variable according to the position of the plunger within the body, wherein the cavity volume contains a first component of the pharmaceutical product;
a vial containing a second component of the pharmaceutical product. The pharmaceutical product may be a mixed pharmaceutical product, made up of at least two parts when reconstituted or mixed

59. A system for reconstituting a pharmaceutical product, comprising:
a syringe comprising a body, a plunger moveable between an initial position and a retracted position relative to the body, a discharge opening, a locking mechanism configured to automatically engage when the plunger is withdrawn to the retracted position to lock the plunger in the retracted position to prevent at least distal movement of the plunger from the retracted position, and a cavity volume defined by the body and plunger, wherein the capacity of the cavity volume is variable according to the position of the plunger within the body, wherein the retracted position is one in which the plunger is retracted proximally relative to the initial position, to provide for an increased cavity volume relative to the cavity volume defined by the body and the plunger in the initial position wherein the cavity volume contains a first component of the pharmaceutical product;
a vial containing a second component of the pharmaceutical product. The pharmaceutical product may be a mixed pharmaceutical product, made up of at least two parts when reconstituted or mixed

60. The system of embodiment 58 or embodiment 59, wherein the syringe is the syringe of any of embodiments 1 to 43.

61. A syringe, comprising:
a body; and
a plunger moveable between an initial position and a retracted position within and relative to the body, the plunger comprising:
   a piston; and
   a flexible seal coupled to a proximal region of the body of the syringe and having an expanded configuration and a contracted configuration, the flexible seal being configured to transition from the expanded configuration to the contracted configuration as the plunger is moved proximally, from the initial position towards the retracted position, and/or configured to expand from the contracted configuration to the expanded configuration as the plunger is moved distally, from the retracted position towards or past the initial position,
wherein the body and the plunger define a cavity volume within the syringe, wherein the capacity of the cavity volume is variable according to the position of the plunger within the body.

62. The syringe of embodiment 61, wherein an entire circumference of the flexible seal is coupled to the body.

63. The syringe of embodiment 61 or 62, wherein the flexible seal is attached to a proximal end cap which is attached to the body at a proximal end of the body. The end cap may comprise a latch receiving component, e.g. aperture or channel, for receiving the latching member of embodiment 9 when the end cap is in place on the syringe body.

64. The syringe of embodiment 63, wherein the proximal end cap is configured for removable attachment to the body. For example, the proximal end cap may be formed with a screw, clip, snap fit or locking fit connection.

65. The syringe of embodiment 63, wherein the proximal end cap is configured for a fixed attachment to the body. The fixed connection may be permanent, for example formed from adhesive or compression fit.

66. The syringe of embodiment 61 to embodiment 65, wherein a central portion of the flexible seal is coupled to a distal end of the piston.

67. The syringe of any one of embodiments 61 to 66, wherein an internal surface of the body at the distal end is rounded, and a distal end of the plunger is rounded.

68. The syringe of any one of embodiments 61 to 67, wherein an internal surface of the body at the distal end is hemi-ellipsoidal, and a distal end of the plunger is hemi-ellipsoidal.

69. The syringe of embodiment 68, wherein an internal surface of the body at the distal end is hemispherical, and a distal end of the plunger is hemispherical.

70. The syringe of any one of embodiments 61 to 69, wherein the plunger is further moveable to a depressed position,
wherein the retracted position is one in which the plunger is retracted proximally relative to the initial position, to provide for an increased cavity volume relative to the cavity volume defined by the body and the plunger in the initial position and the depressed position is one in which the plunger is depressed distally relative to the initial position, to provide for a further decreased cavity volume relative to the cavity volume defined by the body and the plunger in the initial position.

71. The syringe of embodiment 70, wherein the flexible seal conforms to the shape of the distal end of the piston, at least when the plunger is positioned in or between the retracted position and the depressed position.

72. The syringe of embodiment 70 or embodiment 71, wherein an expanded portion of the flexible seal conforms to the shape of an internal surface of the distal end of the body when the plunger is in the depressed position.

73. The syringe of any one of embodiments 61 to 72, further comprising a locking mechanism configured to automatically engage when the plunger is withdrawn to the retracted position to lock the plunger in the retracted position relative to the body, to prevent at least distal movement of the plunger from the retracted position.

74. The syringe of embodiment 73, wherein the locking mechanism comprises a latching member extendable from the plunger to latch against a portion of the body of the syringe when the plunger is moved to the retracted position, thereby locking the plunger in the retracted position relative to the body,
wherein the portion of the body of the syringe against which the latching member latches is proximal of the portion of the body to which the flexible seal is fixed.

75. The syringe of embodiment 73, wherein the locking mechanism comprises a latching member extendable from the body of the syringe to latch against a portion of the plunger when the plunger is in the retracted position, thereby locking the plunger in the retracted position relative to the body,
wherein the latching member extends from the body proximal of the portion of the body to which the flexible seal is fixed.

76. An end cap for attachment to a proximal end of a body of a syringe, the end cap comprising:
a connector adapted for attaching the end cap to the proximal end of the body,
an aperture formed therethrough, sized to movably receive a piston of a plunger;
a flexible seal attachable to the end cap and covering the aperture, the flexible seal having an expanded configuration and a contracted configuration, the flexible seal being configured to transition from the expanded configuration to the contracted configuration within the body of the syringe, when the end cap is attached to the proximal end of the syringe.

77. The syringe of embodiment 76, wherein the flexible seal is configured to stretch from the contracted configuration to the expanded configuration, and relax from the expanded configuration to the contracted configuration.

78. The end cap of embodiment 76 or embodiment 77, wherein the end cap is configured for removable attachment to the body.

79. The end cap of embodiment 76 or embodiment 77, wherein the end cap is configured for fixed attachment to the body.

80. The end cap of any one of embodiments 76 to 79, wherein the flexible seal is attached to or within the end cap.

81. The end cap of any one of embodiments 76 to 79, wherein the flexible seal is configured to be fixed between the end cap and the body, when the end cap is attached to the proximal end of the body.

82. The end cap of any one of embodiments 76 to 81, further comprising a locking mechanism configured to lock the plunger, which is moveable between an initial position and a retracted position proximal of the initial position, in the retracted position.

83. The system of any one of embodiments 58 to 60, wherein the mixed pharmaceutical product is reconstituted Remicade®.

84. The system of any one of embodiments 58 to 60, wherein the mixed pharmaceutical product is Sylvant®.

1. A syringe, comprising:
a body comprising a discharge opening at a distal end and a plunger receiving opening at a proximal end;
a plunger moveable between an initial position and a retracted position within and relative to the body, wherein the retracted position is located proximal to the initial position; and
a locking mechanism configured to lock the plunger in the retracted position relative to the body,
wherein the body and plunger define a cavity volume within the syringe, and
wherein a portion of the plunger creates an air tight seal with the interior of the syringe body, whereby proximal movement of the plunger with respect to the body creates decreased pressure within the cavity volume.

2. (canceled)

3. (canceled)

4. The syringe of claim 1, wherein the plunger is further moveable to a depressed position, wherein the depressed position is located distal to the initial position.

5. The syringe of claim 1, further comprising an automatic retraction stop mechanism configured to prevent the plunger from being moveable to a position proximal of the retracted position.

6. (canceled)

7. The syringe of claim 1, wherein the body comprises a finger flange located on a proximal end of the body, and the locking mechanism comprises a latching member extendable from the plunger and a biasing member configured to extend the latching member laterally outwards of the plunger to latch against the finger flange when the plunger is moved to the retracted position, thereby locking the plunger in the retracted position relative to the body.

8-11. (canceled)

12. The syringe of claim 7, wherein depression of the latching member laterally toward the plunger unlocks the plunger from the retracted position.

13. The syringe of claim 1, wherein the body comprises a finger flange located on a proximal end of the body, and the locking mechanism comprises a latching member extendable from the finger flange and a biasing member configured to extend the latching member laterally towards the plunger to latch against a portion of the plunger when the plunger is in the retracted position, thereby locking the plunger in the retracted position relative to the body.

14. (canceled)

15. The syringe of claim 13, wherein the portion of the plunger comprises an opening through which the latching member is configured to extend when the plunger is withdrawn to the retracted position.

16. (canceled)

17. (canceled)

18. The syringe of claim 13, wherein depression of the latching member laterally outwards of the plunger unlocks the plunger from the retracted position.

19. The syringe of claim 7, wherein the latching member comprises a bar having a proximal end and a distal end, the proximal end being pivotably secured within a proximal portion of the plunger, the distal end being configured to extend laterally outwards of the plunger and to latch against the finger flange.

20. The syringe of claim 7, wherein the plunger further comprises a button movable between an actuated position and a non-actuated position, the button being configured to disengage the latching member from the portion of the body of the syringe when in the actuated position.

21. The syringe of claim 20, wherein the latching member comprises a camming surface and the button is configured, when actuated, to act against the camming surface to move the latching member in a direction laterally inwards of the plunger.

22. The syringe of claim 20, wherein the latching member comprises teeth and the plunger further comprises a gear wheel configured to engage the teeth, wherein actuation of the button is configured to cause rotation of the gear wheel in a direction which drives the latching member to retract in a direction laterally inwards of the plunger.

23. The syringe of claim 20, wherein the button is in the actuated position when the plunger is in the initial position and when the plunger is withdrawn to the retracted position, and upon force from the biasing member, the button is driven to the non-actuated position.

24. The syringe of claim 20, wherein the plunger further comprises an interior member that extends distally from the button and is positioned to be abutted by an end of the biasing member opposite to the latching member when the button is in the non-actuated position, the interior member being configured to move longitudinally through the plunger concurrently with the button, the interior member comprising an opening larger than the biasing member and located at a position corresponding to that of the biasing member when the button is at the actuated position.

25. The syringe of claim 7, wherein the plunger comprises a cylindrical body portion comprising an opening through which the latching member is configured to extend laterally when the plunger is withdrawn to the retracted position and upon force from the biasing member.

26. (canceled)

27. (canceled)

28. The syringe of claim 1, wherein the portion of the plunger that creates an air tight seal with the interior of the syringe body comprises a flexible seal coupled to a proximal region of the body of the syringe and having an expanded configuration and a contracted configuration, the flexible seal being configured to transition from the expanded configuration to the contracted configuration as the plunger is moved proximally, from the initial position towards the retracted position, and/or configured to expand from the contracted configuration to the expanded configuration as the plunger is moved distally, from the retracted position towards or past the initial position.

29. (canceled)

30. The syringe of claim 28, wherein an entire circumference of the flexible seal is coupled to the body.

31. The syringe of claim 28, wherein the flexible seal is coupled to a proximal end cap which is attached to the body at a proximal end of the body.

32. The syringe of claim 31, wherein the proximal end cap is configured for removable attachment to the body.

33. The syringe of claim 31, wherein the proximal end cap is configured for a fixed attachment to the body.

34. The syringe of claim 28, wherein the plunger further comprises a piston, and a central portion of the flexible seal is coupled to a distal end of the piston.

35-79. (canceled)
